(19) United States
(12) Patent Application Publication
(10) Pub. No.: US 2011/0112158 A1
(43) Pub. Date: May 12, 2011

(54) BENZISOXAZOLE ANALOGS AS GLYCOGEN SYNTHASE ACTIVATORS

(76) Inventors: David Robert Bolin, Montclair, NJ (US); Matthew Michael Hamilton, Hackettstown, NJ (US); Lee Apostle McDermott, Aspinwall, PA (US); Yimin Qian, Wayne, NJ (US)

(21) Appl. No.: 12/903,422

(22) Filed: Oct. 13, 2010

Related U.S. Application Data
(60) Provisional application No. 61/260,066, filed on Nov. 11, 2009.

(51) Int. Cl. A61K 31/42; C07D 261/20; C07D 413/12; A61P 3/00

(52) U.S. Cl. 514/379; 548/241

(57) ABSTRACT

Provided herein are compounds of the formula (I):

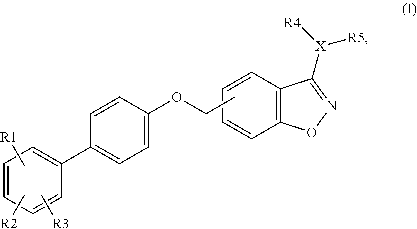

as well as pharmaceutically acceptable salts thereof, wherein the substituents are as those disclosed in the specification. These compounds, and the pharmaceutical compositions containing them, are useful for the treatment of metabolic diseases and disorders such as, for example, type II diabetes mellitus.

### PRIORITY TO RELATED APPLICATION(S)

This application claims the benefit of U.S. Provisional Application No. 61/260,066, filed Nov. 11, 2009, which is hereby incorporated by reference in its entirety.

### FIELD OF THE INVENTION

The invention is directed to compounds, salts and pharmaceutical compositions useful as activators of glycogen synthase for the treatment of metabolic diseases and disorders.

All documents cited or relied upon below are expressly incorporated herein by reference.

### BACKGROUND OF THE INVENTION

Diabetes mellitus is a common and serious disorder, affecting 10 million people in the U.S. [Harris, M. I. Diabetes Care 1998 21 (3S) Supplement, 11C], putting them at increased risk of stroke, heart disease, kidney damage, blindness, and amputation. Diabetes is characterized by decreased insulin secretion and/or an impaired ability of peripheral tissues to respond to insulin, resulting in increased plasma glucose levels. The incidence of diabetes is increasing, and the increase has been associated with increasing obesity and a sedentary life. There are two forms of diabetes: insulin-dependent and non-insulin-dependent, with the great majority of diabetics suffering from the non-insulin-dependent form of the disease, known as type 2 diabetes or non-insulin-dependent diabetes mellitus (NIDDM). Because of the serious consequences, there is an urgent need to control diabetes.

Treatment of NIDDM generally starts with weight loss, a healthy diet and an exercise program. However, these factors are often unable to control the disease, and there are a number of drug treatments available, including insulin, metformin, sulfonylureas, acarbose, and thiazolidinediones. Each of these treatments has disadvantages and there is an ongoing need for new drugs to treat diabetes.

Metformin is an effective agent that reduces fasting plasma glucose levels and enhances the insulin sensitivity of peripheral tissue, mainly through an increase in glycogen synthesis [De Fronzo, R. A. Drugs 1999, 58 Suppl. 1, 29]. Metformin also leads to reductions in the levels of LDL cholesterol and triglycerides [Inzucchi, S. E. JAMA 2002, 287, 360]. However, it loses its effectiveness over a period of years [Turner, R. C. et al. JAMA 1999, 281, 2005].

Thiazolidinediones are activators of the nuclear receptor peroxisome-proliferator activated receptor-gamma. They are effective in reducing blood glucose levels, and their efficacy has been attributed primarily to decreasing insulin resistance in skeletal muscle [Tadayyon, M. and Smith, S. A. Expert Opin. Investig. Drugs 2003, 12, 307]. One disadvantage associated with the use of thiazolidinediones is weight gain.

Sulfonylureas bind to the sulfonylurea receptor on pancreatic beta cells, stimulate insulin secretion, and consequently reduce blood glucose levels. Weight gain is also associated with the use of sulfonylureas [Inzucchi, S. E. JAMA 2002, 287, 360] and, like metformin, they lose efficacy over time [Turner, R. C. et al. JAMA 1999, 281, 2005]. A further problem often encountered in patients treated with sulfonylureas is hypoglycemia [Salas, M. and Caro, J. J. Adv. Drug React. Tox. Rev. 2002, 21, 205-217].

Acarbose is an inhibitor of the enzyme alpha-glucosidase, which breaks down disaccharides and complex carbohydrates in the intestine. It has lower efficacy than metformin or the sulfonylureas, and it causes intestinal discomfort and diarrhea which often lead to the discontinuation of its use [Inzucchi, S. E. JAMA 2002, 287, 360].

Because none of these treatments is effective over the long term without serious side effects, there is a need for new drugs for the treatment of type 2 diabetes.

In skeletal muscle and liver, there are two major pathways of glucose utilization: glycolysis, or oxidative metabolism, where glucose is oxidized to pyruvate; and glycogenesis, or glucose storage, where glucose is stored in the polymeric form glycogen. The key step in the synthesis of glycogen is the addition of the glucose derivative UDP-glucose to the growing glycogen chain, and this step is catalyzed by the enzyme glycogen synthase [Cid, E. et al. J. Biol. Chem. 2000, 275, 33614]. There are two isoforms of glycogen synthase, found in liver [Bai, G. et al. J. Biol. Chem. 1990, 265, 7843] and in other peripheral tissues including muscle [Browner, M. F. et al. Proc. Nat. Acad. Sci. U.S.A. 1989, 86, 1443].

There is clinical and genetic evidence implicating both forms of glycogen synthase in metabolic diseases such as type 2 diabetes and cardiovascular disease. Both basal and insulin-stimulated glycogen synthase activity in muscle cells from diabetic subjects were significantly lower than in cells from lean non-diabetic subjects [Henry, R. R. et al. J. Clin. Invest. 1996, 98, 1231-1236; Nikoulina, S. E. et al. J. Clin. Enocrinol. Metab. 2001, 86, 4307-4314]. Furthermore, several studies have shown that levels of muscle [Eriksson, J. et al. N. Engl. J. Mod. 1989, 331, 337; Schulman, R. G. et al. N. Engl. J. Med. 1990, 332, 223; Thorburn, A. W. et al. J. Clin. Invest. 1991, 87, 489] and liver [Krssak, M. et. al. Diabetes 2004, 53, 3048] glycogen are lower in diabetic patients than in control subjects. In addition, genetic studies have shown associations in several populations between type 2 diabetes and/or cardiovascular disease and mutation/deletion in the GYS1 gene encoding the muscle isoform of glycogen synthase [Orhu-Melander, M. et al. Diabetes 1999, 48, 918; Fredriksson, J. et. al. PLoS ONE 2007, 3, e285; Kolhberg G. et. al. N. Engl. J. Med. 2007, 357, 1507]. Patients lacking GYS2 encoding the liver isoform of glycogen synthase, suffer from fasting ketotic hypoglycemia and postprandial hyperglycemia, hyperlactanemia and hyperlipidemia, supporting the essential role of liver GS in maintaining normal nutrient metabolism. [Weinstein, D. A. et. al. Mol. Genetics and Metabolism, 2006, 87, 284]

Glycogen synthase is subject to complex regulation, involving phosphorylation in at least nine sites [Lawrence, J. C., Jr. and Roach, P. J. Diabetes 1997, 46, 541]. The dephosphorylated form of the enzyme is active. Glycogen synthase is phosphorylated by a number of enzymes of which glycogen synthase kinase 3β (GSK3β) is the best understood [Tadayyon, M. and Smith, S. A. Expert Opin. Investig. Drugs 2003, 12, 307], and glycogen synthase is dephosphorylated by protein phosphatase type I (PP1) and protein phosphatase type 2A (PP2A). In addition, glycogen synthase is regulated by an endogenous ligand, glucose-6-phosphate which allosterically stimulates the activity of glycogen synthase by causing a change in the conformation of the enzyme that renders it more susceptible to dephosphorylation by the protein phosphatases to the active form of the enzyme [Gomis, R. R. et al. J. Biol. Chem. 2002, 277, 23246].

Several mechanisms have been proposed for the effect of insulin in reducing blood glucose levels, each resulting in an increase in the storage of glucose as glycogen. First, glucose uptake is increased through recruitment of the glucose transporter GLUT4 to the plasma membrane [Holman, G. D. and Kasuga, M. Diabetologia 1997, 40, 991]. Second, there is an increase in the concentration of glucose-6-phosphate, the allosteric activator of glycogen synthase [Villar-Palasi, C. and Guinovart, J. J. FASEB J. 1997, 11, 544]. Third, a kinase cascade beginning with the tyrosine kinase activity of the insulin receptor results in the phosphorylation and inactivation of GSK3β, thereby preventing the deactivation of glycogen synthase [Cohen, P. Biochem. Soc. Trans. 1993, 21, 555; Yeaman, S. J. Biochem. Soc. Trans. 2001, 29, 537].

Because a significant decrease in the activity of glycogen synthase has been found in diabetic patients, and because of its key role in glucose utilization, the activation of the enzyme glycogen synthase holds therapeutic promise for the treatment of metabolic diseases such as type 2 diabetes and cardiovascular diseases. The only known allosteric activators of the enzyme are glucose-6-phosphate [Leloir, L. F. et al. Arch. Biochem. Biophys. 1959, 81, 508] and glucosamine-6-phosphate [Virkamaki, A. and Yki-Jarvinen, H. Diabetes 1999, 48, 1101].

The following biaryloxymethylarenecarboxylic acids are reported to be commercially available from Otava, Toronto, Canada, Akos Consulting & Solutions, Steinen, Germany or Princeton BioMolecular Research, Monmouth Junction, N.J.: 4-(biphenyl-4-yloxymethyl)-benzoic acid, 3-(biphenyl-4-yloxymethyl)-benzoic acid, [4-(biphenyl-4-yloxymethyl)-phenyl]-acetic acid, [4-(4′-methyl-biphenyl-4-yloxymethyl)-phenyl]-acetic acid, 4-(4′-methyl-biphenyl-4-yloxymethyl)-benzoic acid, 3-(3-bromo-biphenyl-4-yloxymethyl)-benzoic acid, [4-(3-bromo-biphenyl-4-yloxymethyl)-phenyl]-acetic acid, 2-(4′-methyl-biphenyl-4-yloxymethyl)-benzoic acid, 5-(biphenyl-4-yloxymethyl)-furan-2-carboxylic acid, 5-(4′-methyl-biphenyl-4-yloxymethyl)-furan-2-carboxylic acid, 5-(3-bromo-biphenyl-4-yloxymethyl)-furan-2-carboxylic acid, 4-(biphenyl-4-yloxymethyl)-5-methyl-furan-2-carboxylic acid, 5-methyl-4-(4′-methyl-biphenyl-4-yloxymethyl)-furan-2-carboxylic acid, 4-(3-bromo-biphenyl-4-yloxymethyl)-5-methyl-furan-2-carboxylic acid, 2-(biphenyl-4-yloxymethyl)-4-methyl-thiazole-5-carboxylic acid, [2-(biphenyl-4-yloxymethyl)-thiazol-4-yl]-acetic acid, [2-(4′-methyl-biphenyl-4-yloxymethyl)-thiazol-4-yl]-acetic acid and [5-(biphenyl-4-yloxymethyl)-[1,3,4]oxadiazol-2-yl]-acetic acid.

Some biaryloxymethylarenecarboxylic acids are known in the art. However, none of these known compounds have been associated with either the treatment of diseases mediated by the activation of the glycogen synthase enzyme or to any pharmaceutical composition for the treatment of diseases mediated by the activation of the glycogen synthase enzyme. Andersen, H. S. et al. WO 9740017 discloses the structure and synthetic route to 3-(biphenyl-4-yloxymethyl)-benzoic acid as an intermediate in the synthesis of SH2 inhibitors. Winkelmann, E. et al. DE 2842243 discloses 5-(biphenyl-4-yloxymethyl)-thiophene-2-carboxylic acid as a hypolipemic agent. Mueller, T. et al. DE 4142514 discloses 2-(biphenyl-3-yloxymethyl)-benzoic acid as a fungicide. Ghosh, S. S. et al. WO 2004058679 discloses biaryloxymethylarene acids as ligands of adenine nucleoside translocase. Van Zandt, M. C. WO 2008033455 discloses biphenyl and heteroarylphenyl derivatives as protein phosphatase-1B inhibitors.

Glycogen synthase activators and stimulators of glycogen production have been reported. Chu, C. A et al. US 20040266856 discloses biaryoxymethylarenecarboxylic acids as glycogen synthase activators. Chu, C. A. WO 2005000781 discloses biaryloxymethylarene carboxylic acids as activators of glycogen synthase. Yang, S-P. and Huang, Y. US 20050095219 discloses hyaluronic acid compounds that stimulate glycogen production. Gillespie, P. et al. WO 2005075468 discloses biaryoxymethylarene carboxylic acids as glycogen synthase activators. Gillespie, P. et al. WO 2006058648 discloses biaryoxymethylarene carboxylic acids as glycogen synthase activators. Bucala, R. et al. WO 2007044622 discloses macrophage migration inhibitory factor agonists that stimulate glycogen production.

### SUMMARY OF THE INVENTION

The present invention is directed to compounds of the formula I:

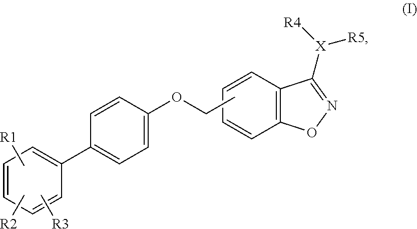

as well as pharmaceutically acceptable salts thereof, pharmaceutical compositions containing them and to methods of treating diseases and disorders. The compounds and compositions disclosed herein are glycogen synthase activators and are useful for the treatment of metabolic diseases and disorders, preferably diabetes mellitus, more preferably type II diabetes mellitus.

### DETAILED DESCRIPTION OF THE INVENTION

In an embodiment of the present invention, provided is a compound of formula (I):

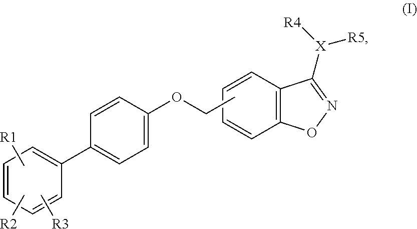

wherein:
X is O or N;
R1, R2, R3, independently of each other, is halogen, alkoxy or unsubstituted lower alkyl; and
one of R4 or R5 is hydrogen or lower alkyl, unsubstituted or mono-, bi- or tri-substituted with lower alkyl, —COOH, aryl or alkoxy, and the other is absent, when X is O, or
one of R4 or R5 is hydrogen, cycloalkyl or lower alkyl substituted with —COOH or alkoxy, and the other is hydrogen, unsubstituted lower alkyl, or lower alkyl substituted with alkoxy, cycloalkyl or heterocycloalkyl, when X is N,
   or a pharmaceutically acceptable salt thereof.

In another embodiment, X is O. In further embodiment, X is N.

Preferably, R1 is F or Cl.

Preferably, R2 is F or Cl.

Preferably, R3 is F, Cl, methyl or methoxy.

Preferably, when X is O, then one of R4 or R5 is hydrogen or lower alkyl, unsubstituted or mono-, bi- or tri-substituted with lower alkyl, —COOH, aryl or alkoxy, and the other is absent.

Preferably, when X is N, then one of R4 or R5 is hydrogen, cycloalkyl or lower alkyl substituted with —COOH or alkoxy, and the other is hydrogen, unsubstituted lower alkyl, or lower alkyl substituted with alkoxy, cycloalkyl or heterocycloalkyl.

Preferably, when X is O, then one of R4 or R5 is:

H,

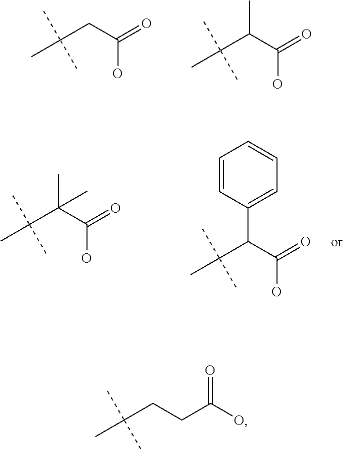

and the other is absent.

Preferably, when X is N, then one of R4 or R5 is H or CH₂COOH and the other is:

H, methyl, ethyl, cyclopropylmethyl,

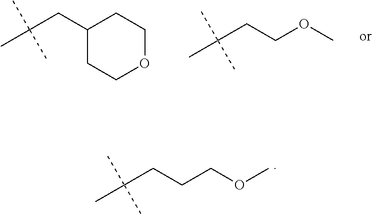

Preferably, the compound according to formula (I) is:
5-(4′,5′-Difluoro-2′-methoxy-biphenyl-4-yloxymethyl)-benzo[d]isoxazol-3-ylamine;
([5-(4′,5′-Difluoro-2′-methoxy-biphenyl-4-yloxymethyl)-benzo[d]isoxazol-3-yl]-methyl-amine;
{[5-(4′,5′-Difluoro-2′-methoxy-biphenyl-4-yloxymethyl)-benzo[d]isoxazol-3-yl]-methyl-amino}-acetic acid;
5-(4′,5′-Difluoro-2′-methoxy-biphenyl-4-yloxymethyl)-benzo[d]isoxazol-3-ol;
[5-(4′,5′-Difluoro-2′-methoxy-biphenyl-4-yloxymethyl)-benzo[d]isoxazol-3-yloxy]-acetic acid;
2-[5-(4′,5′-Difluoro-2′-methoxy-biphenyl-4-yloxymethyl)-benzo[d]isoxazol-3-yloxy]-2-methyl-propionic acid;
2-[5-(4′,5′-Difluoro-2′-methoxy-biphenyl-4-yloxymethyl)-benzo[d]isoxazol-3-yloxy]-propionic acid;
[5-(4′,5′-Difluoro-2′-methoxy-biphenyl-4-yloxymethyl)-benzo[d]isoxazol-3-yloxy]-phenyl-acetic acid;
3-[5-(4′,5′-Difluoro-2′-methoxy-biphenyl-4-yloxymethyl)-benzo[d]isoxazol-3-yloxy]-propionic acid;
7-(2′,4′,5′-Trifluoro-biphenyl-4-yl-oxymethyl)-benzo[d]isoxazol-3-ol;
7-(4′,5′-Difluoro-2′-methoxy-biphenyl-4-yloxymethyl)-benzo[d]isoxazol-3-ol;
7-(2′-Chloro-4′,5′-difluoro-biphenyl-4-yloxymethyl)-benzo[d]isoxazol-3-ol;
7-(4′,5′-Difluoro-2′-methyl-biphenyl-4-yloxymethyl)-benzo[d]isoxazol-3-ol;
[5-(4′,5′-Difluoro-2′-methoxy-biphenyl-4-yloxymethyl)-benzo[d]isoxazol-3-yl]-(2-methoxy-ethyl)-amino]-acetic acid;
{[5-(4′,5′-Difluoro-2′-methoxy-biphenyl-4-yloxymethyl)-benzo[d]isoxazol-3-yl]-(3-methoxy-propyl)-amino}-acetic acid;
{[5-(4′,5′-Difluoro-2′-methoxy-biphenyl-4-yloxymethyl)-benzo[d]isoxazol-3-yl]-ethyl-amino}-acetic acid;
{Cyclopropylmethyl-[5-(4′,5′-difluoro-2′-methoxy-biphenyl-4-yloxymethyl)-benzo[d]isoxazol-3-yl]-amino}-acetic acid;
{[5-(4′,5′-Difluoro-2′-methoxy-biphenyl-4-yloxymethyl)-benzo[d]isoxazol-3-yl]-(tetrahydro-pyran-4-ylmethyl)-amino}-acetic acid;
{[7-(4′,5′-Difluoro-2′-methoxy-biphenyl-4-yloxymethyl)-benzo[d]isoxazol-3-yl]-methyl-amino}-acetic acid; or
5-(2′,4′,5′-Trifluoro-biphenyl-4-yloxymethyl)-benzo[d]isoxazol-3-ol.

In another preferred embodiment, provided is a pharmaceutical composition, comprising a therapeutically effective amount of a compound according to formula (I) and a pharmaceutically acceptable carrier and/or adjuvant.

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described.

As used herein, the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “cycloalkyl” refers to a monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, bornyl, adamantyl, indenyl and the like. In a preferred embodiment, the “cycloalkyl” moieties can optionally be substituted with one, two, three or four substituents with the understanding that said substituents are not, in turn, substituted further unless indicated otherwise in the Examples or claims below. Each substituent can independently be, for example, alkyl, alkoxy, halogen, amino, hydroxyl or oxygen (O═) unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl.

The term “heterocycloalkyl” denotes a mono- or polycyclic alkyl ring, wherein one, two or three of the carbon ring atoms is replaced by a heteroatom such as N, O or S. Examples of heterocycloalkyl groups include, but are not limited to, pyranyl, morpholinyl, thiomorpholinyl, piperazinyl, piperidinyl, pyrrolidinyl, tetrahydropyranyl, tetrahydrofuranyl, 1,3-dioxanyl and the like. The heterocycloalkyl groups may be unsubstituted or substituted and attachment may be through their carbon frame or through their heteroatom(s) where appropriate, with the understanding that said substituents are not, in turn, substituted further unless indicated otherwise in the Examples or claims below.

The term “lower alkyl”, alone or in combination with other groups, refers to a branched or straight-chain alkyl radical of one to nine carbon atoms, preferably one to six carbon atoms, most preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “aryl” refers to an aromatic mono- or polycarbocyclic radical of 6 to 12 carbon atoms having at least one aromatic ring. Examples of such groups include, but are not limited to, phenyl, napthyl. 1,2,3,4-tetrahydronaphthalene, 1,2-dihydronaphthalene, indanyl, 1H-indenyl and the like.

The alkyl, lower alkyl and aryl groups may be substituted or unsubstituted. When substituted, there will generally be, for example, 1 to 4 substituents present, with the understanding that said substituents are not, in turn, substituted further unless indicated otherwise in the Examples or claims below. Substituents may include, for example, alkoxy, lower alkyl, cycloalkyl, aryl, —COOH and heterocycloalkyl groups.

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. The heteroaryl group may be substituted independently with one, two, or three substituents, with the understanding that said substituents are not, in turn, substituted further unless indicated otherwise in the Examples or claims below. Substituents may include, for example, halogen and lower alkyl groups.

As used herein, the term “alkoxy” means alkyl-O—; and “alkoyl” means alkyl-CO—. Alkoxy substituent groups or alkoxy-containing substituent groups may be substituted by, for example, one or more alkyl groups with the understanding that said substituents are not, in turn, substituted further unless indicated otherwise in the Examples or claims below.

As used herein, the term “halogen” means a fluorine, chlorine, bromine or iodine radical, preferably a fluorine, chlorine or bromine radical, and more preferably a fluorine or chlorine radical.

Compounds of formula (I) can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with chiral adsorbents or eluant). The invention embraces all of these forms.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Particularly preferred are fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminium salts.

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. Preferably, the therapeutically effective amount is in an amount of from about 1 mg to about 500 mg per day.

It will be appreciated, that the compounds of general formula (I) in this invention may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

Compounds of the present invention can be prepared beginning with commercially available starting materials and utilizing general synthetic techniques and procedures known to those skilled in the art. Outlined below are reaction schemes suitable for preparing such compounds. Further exemplification can be found in the specific Examples detailed below.

Chemicals may be purchased from companies such as for example Aldrich, Argonaut Technologies, VWR and Lancaster. Chromatography supplies and equipment may be purchased from such companies as for example AnaLogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography.

Definitions as used herein:
GS is glycogen synthase,
THF is tetrahydrofuran,
DMF is N,N-dimethylformamide,
DMA is N,N-dimethylacetamide,
DMSO is dimethylsulfoxide,
DCM is dichloromethane,
DME is dimethoxyethane,
MeOH is methanol,
EtOH is ethanol,
NaOH is sodium hydroxide,
TFA is 1,1,1-trifluoroacetatic acid,
HOBT is 1-hydroxybenzotriazole,
PyBroP is bromotripyrrolidinophosphonium hexafluorophosphate,
EDCI is 1-[3-(dimethylamino)propyl]-3-ethylcarbodiimide hydrochloride,
DIPEA is diisopropylethylamine,
Boc is tert-butyloxycarbonyl,
NBS is N-bromosuccinimde,
DMAP is N,N-dimethylamino-pyridine,
DEAD is diethyl azodicarboxylate,
V65 is 2,2′-azobis(2.4-dimethyl valeronitrile),
Brine is saturated aqueous sodium chloride solution,
TLC is thin layer chromatography,
HR-MS is high resolution mass spectrometry,
LC-MS is liquid chromatographic mass spectrometry,
RT is room or ambient temperature.
ES is electron spray mass spectrometry

The preparation of substituted biphenylphenols is described in Scheme 1, below. Commercially available phenylboronic acid (i) can be coupled with 4-iodophenol under palladium catalysis conditions to form the bi-aryl-phenol (ii), where R1, R2 and R3 can be fluoro, chloro, methyl or methoxy groups. Alternatively, the required biphenylphenol (iv) can also be prepared through the coupling of 4-hydroxy-arylboronic acid with the corresponding arylbromide under palladium catalysis conditions (Scheme 1). Non-commercially available arylbromides (v) can be prepared through aromatic bromination.

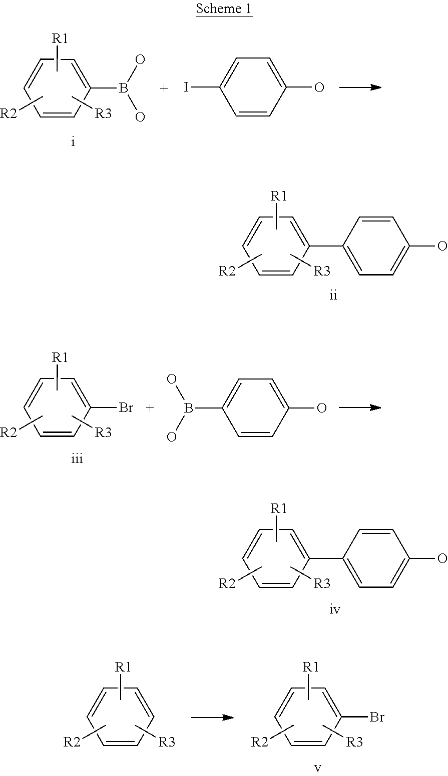

The preparation of substituted 3-amino-benzisoxazoles (xi) is shown in Scheme 2, below. Fluoro-methyl benzonitrile (vi) can be reacted with acetone oxime under basic conditions, preferably potassium t-butoxide, to give benzisoxazole (vii). The amine of vii may be protected using di-tert-butyl-dicarbonate to give the N,N-di-Boc-protected compound viii. Compound viii may be brominated with N-bromo-succinimide in CCl₄ in the presence of a radical initiator, such as 2,2′-azobis(2-methylpropionitrile) to give ix. The bromomethyl-benzisoxazole (ix) can be alkylated with a substituted bi-aryl-phenol under basic conditions to form a substituted-bi-phenyloxymethyl-benzisoxazole (x), which may be deprotected under acidic conditions, such as HCl or TFA in various solvents to give substituted 3-amino-benzisoxazoles (xi), where R1, R2 and R3 may be fluoro, chloro, methyl or methoxy.

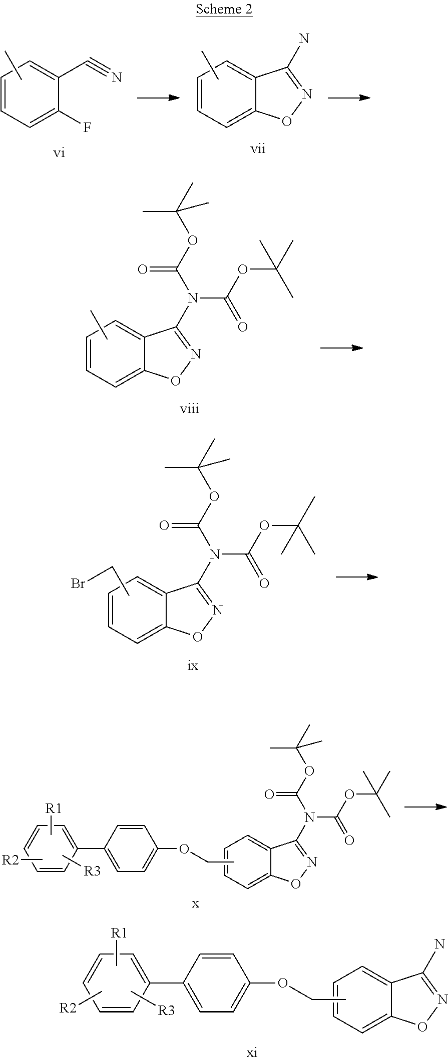

N-Alkyl-3-amino-benzisoxazoles (xiv) may be prepared as shown in Scheme 3, below. Substituted-bi-phenyloxymethyl-benzisoxazole (x) may be mono-deprotected with hydrazine hydrate. The Boc-amine may be then alkylated under basic conditions with various alkylating reagents to give compound xiii. The Boc protecting group can be removed under acidic conditions, preferably TFA/CH2Cl2 to give xiv, where R1, R2 and R3 may be fluoro, chloro, methyl or methoxy and R4 may be a lower alkyl, alkoxy-alkyl or cycloalkyl.

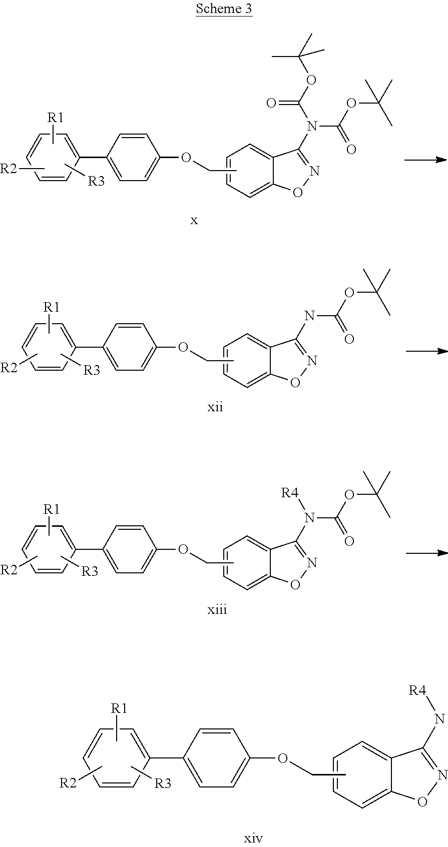

As shown in Scheme 4, below, substituted-alkyl-amine xiv may be treated with a bromoacetate under basic conditions, such as lithium bis(trimethylsilyl)amide or cesium carbonate to give ester xv, which may be hydrolyzed to acid xvi, where R1, R2 and R3 may be fluoro, chloro, methyl or methoxy and R4 may be a lower alkyl, alkoxy-alkyl or cycloalkyl.

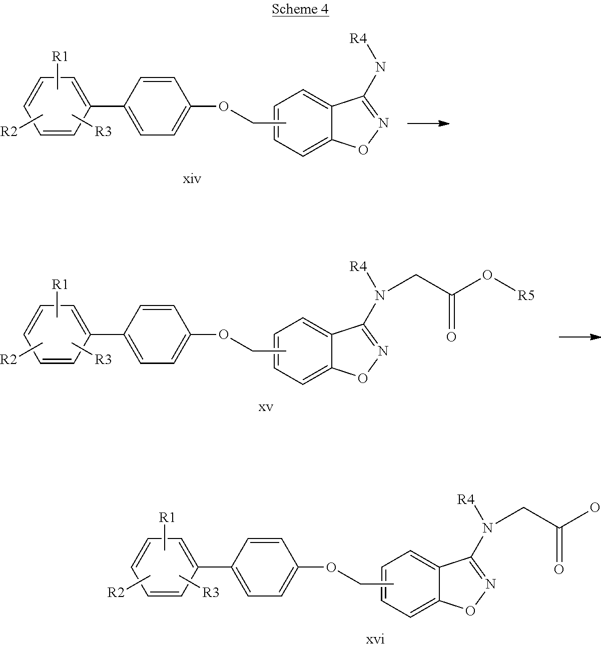

substituted-3-hydroxy-benzisoxazoles xxiv may be prepared as in Scheme 5, below. 5-Methyl-salicylic acid methyl ester and hydroxylamine/H₂O can give dihydroxy-5-methyl-benzamide, xvii. Treatment of this compound with carbonyl-diimidazole and heating can yield 3-hydroxy-benzisoxazole xix. The hydroxyl group can be protected with groups such as tetrahydropyran. Compound xx may be brominated with N-bromo-succinimide in CCl₄ in the presence of a radical initiator, such as 2,2′-azobis(2-methylpropionitrile) to give xxi. The bromomethyl-benzisoxazole (xxi) can be alkylated with a substituted bi-aryl-phenol under basic conditions to form a substituted-bi-phenyloxymethyl-benzisoxazole (xxii), which may be deprotected under acidic conditions, such as HCl or TFA in various solvents to give substituted 3-amino-benzisoxazoles (xxiii), where R1, R2 and R3 may be fluoro, chloro, methyl or methoxy. The hydroxyl group of xxiii may be alkylated under basic conditions. In the case of alkyl esters, the esters may be hydrolysed under basic conditions, such as with LiOH.H₂O to give compound xxiv, where R1, R2 and R3 may be fluoro, chloro, methyl or methoxy and R6 may be lower alkyl and alkyl- or aryl-substituted lower alkyl acids.

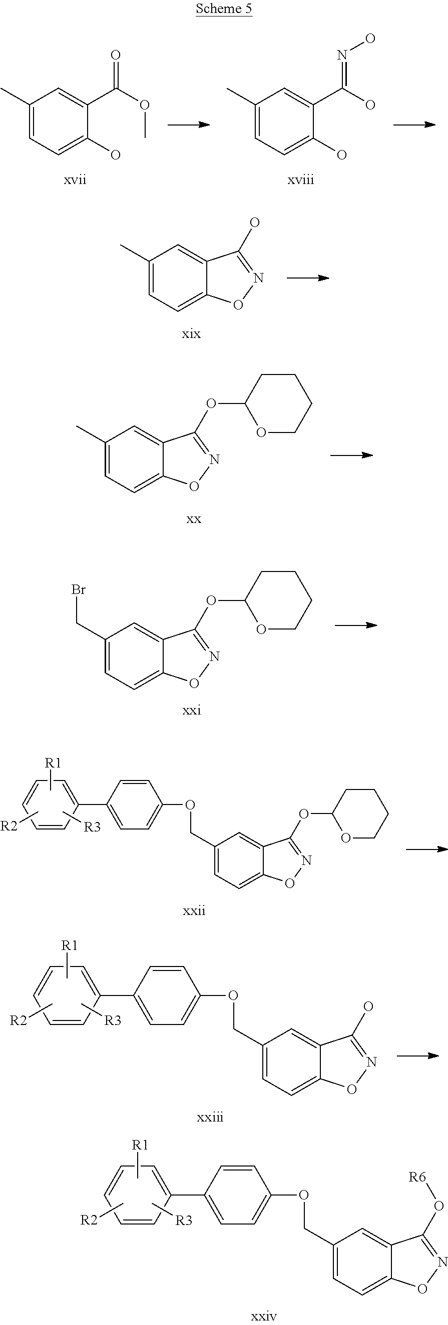

The preparation of substituted 3-hydroxy-benzisoxazoles is shown in Scheme 6. The hydroxyl group of 3-hydroxy-benzisoxazole xxv can be protected with groups such as trityl. Compound xxvi may be brominated with N-bromo-succinimide in CCl4 in the presence of a radical initiator, such as 2,2′-azobis(2-methylpropionitrile) to give xxvii. The bromomethyl-benzisoxazole (xxvii) can be alkylated with a substituted bi-aryl-phenol under basic conditions to form a substituted-bi-phenyloxymethyl-benzisoxazole (xxviii), which may be deprotected under acidic conditions, such as HCl or TFA in various solvents to give substituted 3-hydroxy-benzisoxazoles (xxix), where R1, R2 and R3 may be fluoro, chloro, methyl or methoxy.

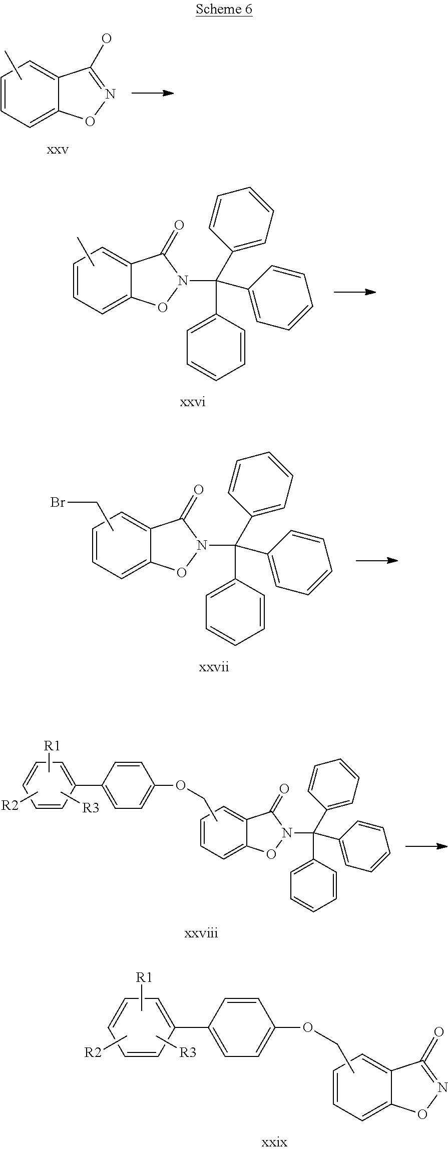

The invention will now be further described in the Examples below, which are intended as an illustration only and do not limit the scope of the invention.

### EXAMPLES

### Part I: Preparation of Preferred Intermediates

### 4′,5′-Difluoro-2′-methoxy-biphenyl-4-ol

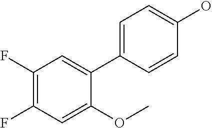

4,5-Difluoro-2-methoxyphenyl-boronic acid (8.8 g, 46.82 mmol) and 4-iodophenol (6.86 g, 31.21 mmol) were suspended in 165 ml of DMF. Water (40 mL) was added and the mixture was degassed with argon. Finely ground potassium carbonate (13 g, 93.63 mmol) and tetrakis(triphenylphosphine) palladium(0) (1.5 g, 1.29 mmol) were added. The reaction was stirred at 80-85° C. for 1 hr under argon and cooled. The mixture was diluted with ethyl acetate and water. The organic layer was washed with brine, dried and solvents were evaporated. The crude product was purified by flash chromatography, eluting with 0-8% ethyl acetate in hexanes to yield 4′,5′-difluoro-2′-methoxy-biphenyl-4-ol (6.58 g, 89.3%). LR-MS (ES) calculated for C13H10F2O2, 236.22. found m/z 235 [M−H].

### 4′,5′-Difluoro-2′-methyl-biphenyl-4-ol

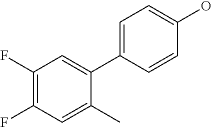

To a stirred solution of 3,4-difluorotoluene (30 g, 234.1 mmol) and iron powder (0.784 g, 14.05 mmol) in methylene chloride (140 mL) in an ice bath was added bromine (12 mL, 155 mmol) slowly over 6.5 hrs. The reaction mixture was warmed to RT and stirred for an additional 60 hrs. The mixture was washed with water (200 mL), 10% Na2SO3 (3×150 mL), saturated (NaHCO3 (100 mL) and saturated NaCl. The organic layer was dried over MgSO4, filtered and concentrated in vacuo to give 40 g of crude 1-bromo-4,5-difluoro-2-methyl-benzene.

Bromo-4,5-difluoro-2-methyl-benzene (30 g, 145 mmol) and 4-hydroxyphenyl-boronic acid (20.99 g, 152.2 mmol) were suspended in 725 ml of dioxane. The mixture was degassed with argon. Potassium carbonate (23.17 g, 166 mmol) in 72.5 ml water solution was added and after stirring for 10 minutes, bis(tricyclohexylphosphine)palladium(0) (2.4 g, 3.62 mmol) was added. The reaction was stirred at 80-85° C. for 19 hrs and concentrated. The mixture was diluted with ethyl acetate and water. The organic layer was washed with brine, dried and solvents were evaporated. The crude product was purified by flash chromatography, eluting with 0-7% ethyl acetate in hexanes to obtain the crude product which was further recrystallized from hexane to afford 4′,5′-difluoro-2′-methyl-biphenyl-4-ol (23.76 g, 74.5%). LR-MS (ES) calculated for C13H10F2O, 220.22. found m/z 219 [M−H].

### 2′,4′,5′-Trifluoro-biphenyl-4-ol

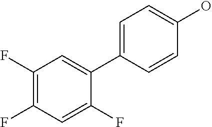

A mixture of 2,4,6-trifluorophenylboronic acid (43.8 g, 249.2 mmol), 4-iodophenol (50 g, 226.5 mmol), potassium carbonate (78 g, 556.3 mmol), Pd (dppf)Cl₂ methylene chloride complex (5.5 g, 6.8 mmol), DMF (150 mL), and water (38 mL) was degassed, purged with nitrogen, and heated at 50° C. overnight. The mixture was then diluted with EtOAc and water, acidified with conc. HCl under cooling with ice-water bath, stirred with charcoal, and filtered through Celite. The organic layer was separated, washed with water and brine, dried over sodium sulfate, filtered, and evaporated to afford a deep red oily crude product. The crude product in EtOAc was passed through silica gel to give light brown solid product (38 g, 75%). LC-MS (ES) calculated for C12H7F3O, 224.18. found m/z 224 [M+H].

### 2′-Chloro-4′,5′-difluoro-biphenyl-4-ol

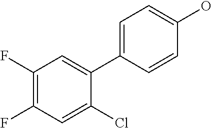

4-Chloro-1,2-difluoro-benzene (12 g, 80.79 mmol) and iron (4.524 g, 80.79 mmol) were mixed with bromine (4.57 mL, 88.87 mmol) and the flask was wrapped with aluminum foil. After stirring at room temperature for 30 min, the mixture was heated on an oil bath at 72° C. overnight. The mixture was cooled to room temperature and aqueous HCl solution (1.0N, 45 ml) was added. The reaction mixture was extracted with methylene chloride and the organic phase was washed with brine, dried and concentrated. The resulting oil was purified using an ISCO (220 g) column chromatography, eluting with hexanes to obtain 1-bromo-2-chloro-4,5-difluoro-benzene as an oil. (8.1 g, 44.1%). ¹H NMR (300 MHz, CDCl₃) δ ppm 7.47 (t, J=8.6 Hz, 1H), 7.33 (dd, J=9.5, 7.4 Hz, 1H).

Bromo-2-chloro-4,5-difluoro-benzene (5.9 g, 25.95 mmol) and 4-hydroxyphenyl-boronic acid (4.29 g, 31.13 mmol) were suspended in 138 ml of dioxane. Potassium carbonate (4.483 g, 32.43 mmol) in 40 ml water solution was added and after stirring for 10 minutes, bis(tricyclohexylphosphine)palladium(0) (530 mg, 0.779 mmol) was added. The reaction was stirred at 85° C. for 4 hrs and concentrated. The mixture was diluted with ethyl acetate and water. The organic layer was washed with brine, dried and solvents were evaporated. The crude product was purified using an ISCO (120 g) column chromatography, eluting with 5-20% ethyl acetate in hexanes to obtain the crude product which was further recrystallized from hexane to afford 2′-chloro-4′,5′-difluoro-biphenyl-4-ol as a white solid (4.6 g, 73.7%). ¹H NMR (300 MHz, DMSO-d6) δ ppm 9.71 (s, 1H), 7.99 (dd, J=10.6, 7.5 Hz, 1H), 7.51 (dd, J=11.5, 8.8 Hz, 1H), 7.25 (d, J=8.7 Hz, 2H), 6.84 (d, J=8.7 Hz, 2H). LR-MS (ES) calculated for C12H7ClF2O, 240.64. found m/z 239 [M−H].

### 5-Bromomethyl-2-trityl-benzo[d] isoxazol-3-one

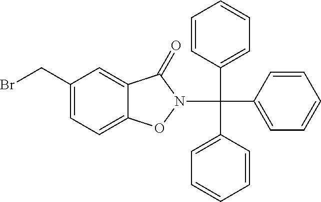

To a solution of 5-methyl-benzoisoxazol-3-ol (8.0 g, 53.7 mmol) in methylene chloride (60 mL) was added trityl chloride (14.0 g, 50.0 mmol) and pyridine (4.0 g, 50.6 mmol). The mixture was refluxed for 1 hr and then extracted with methylene chloride and water. The organic layer was extracted with diluted (0.5N) aqueous sodium hydroxide solution and washed with water and brine. The solution was dried over sodium sulfate and solvents were evaporated. The residue was purified through ISCO flash column chromatography using ethyl acetate in hexanes (330 g silica gel, 0% to 40% linear gradient) to give 5-methyl-2-trityl-benzoisoxazol-3-one as a white solid (15.6 g, 73.4%). ¹H NMR(CHLOROFORM-d) δ ppm 7.52 (d, J=7.8 Hz, 6H), 7.40 (s, 1H), 7.18-7.36 (m, 10H), 6.99 (d, J=8.5 Hz, 1H), 2.34 (s, 3H)

To a solution of 5-methyl-2-trityl-benzoisoxazol-3-one (7.82 g, 20 mmol) in methylene chloride (100 mL) was added NBS (3.91 g, 21.96 mmol) and V65 (250 mg, 1 mmol). The mixture was refluxed for 15 hrs. The cooled mixture was treated with water (60 mL) and the organic layer was separated. The organic layer was washed with brine and sodium bicarbonate solution. The solution was dried over sodium sulfate and filtered. Solvents were evaporated and the residue was purified through ISCO flash column chromatography using ethyl acetate in hexanes (300 g silica gel, 0% to 25% linear gradient) to give a solid as 5-bromomethyl-2-trityl-benzo[d]isoxazol-3-one (6.88 g, 73%). ¹H NMR (DMSO-d₆) δ ppm 7.73 (dd, J=9.0, 1.8 Hz, 1H), 7.69 (s, 1H), 7.47 (d, J=7.2 Hz, 6H), 7.40 (d, J=9.0 Hz, 1H), 7.16-7.36 (m, 9H), 4.75 (s, 2H)

### Part II: Preparation of Preferred Embodiments of the Invention

### Example 1

### 5-(4′,5′-Difluoro-2′-methoxy-biphenyl-4-yloxymethyl)-benzo[d]isoxazol-3-ylamine

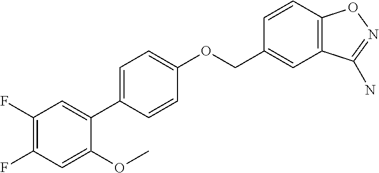

Potassium t-butoxide (4.57 g, 40.7 mmol) was suspended in 40 mL THF. Acetone oxime (2.97 g, 40.6 mmol) was added followed by the addition of 2-fluoro-5 methyl benzonitrile (5 g, 3.7 mmol) in 30 mL THF. The mixture was stirred at RT for 4 hrs and then refluxed overnight. The reaction mixture was worked up as described in WO2007109459 and US2007063893. The solid residue was heated with 80 mL EtOH, 53 mL H₂O and 27 mL 37% HCl to reflux for 2 hrs. The mixture was partitioned between ethyl acetate and H₂O, which was made basic to pH 12. The ethyl acetate layer was separated and the aqueous layer was extracted with ethyl acetate. The combined organic layers were evaporated to yield 5-methyl-benzo[d]isoxazol-3-ylamine as a brown residue.

The crude 5-methyl-benzo[d]isoxazol-3-ylamine was dissolved in 60 mL CH₂Cl₂. To this solution was added di-tert-butyl-dicarbonate (14.6 g, 66.8 mmol) and a catalytic amount of DMAP. The reaction mixture was stirred at RT overnight. The mixture was concentrated in vacuo to about 30 mL volume, treated with hexanes and filtered to yield 4.8 g of N,N-di-Boc-5-methyl-benzo[d]isoxazol-3-ylamine as a solid.

The crude N,N-di-Boc-5-methyl-benzo[d]isoxazol-3-ylamine was dissolved in 35 mL CCl₄. N-bromo-succinimide (0.5 g, 2.87 mmol) and 2,2′-azobis(2-methylpropionitrile) (50 mg, 0.3 mmol) were added to the solution and refluxed for 3 hrs. The reaction was cooled, concentrated in vacuo. The crude product was purified by flash chromatography with 0-3% Et₂O in hexanes to yield N,N-di-Boc-5-bromomethyl-benzo[d]isoxazol-3-ylamine.

To a mixture of THF and DMF (4/1) was added N,N-di-Boc-5-bromomethyl-benzo[d]isoxazol-3-ylamine (0.36 g, 0.847 mmol), 3-(4′,5′-difluoro-2′-methoxy-biphenyl-4-ol (0.2 g, 0.847 mmol) and potassium carbonate (0.12 g, 0.847 mmol). The reaction mixture was stirred overnight and after an aqueous/ethyl acetate workup, the crude product was purified by flash chromatography with a 0-30% ethyl acetate in hexanes gradient to yield N,N-di-Boc-5-(4′,5′-difluoro-2′-methoxy-biphenyl-4-yloxymethyl)-benzo[d]isoxazol-3-ylamine. LC-MS (ES) calculated for C18H24N2O5, 348.40. found m/z 349 [M+H]⁺.

N,N-di-Boc-5-(4′,5′-difluoro-2′-methoxy-biphenyl-4-yloxymethyl)-benzo[d]isoxazol-3-ylamine was treated with 10 mL 10% TFA in CH₂Cl₂ for 1.25 hrs. The mixture was then partitioned between ethyl acetate and H₂O which was made basic to pH 10 with NaOH. The organic layer was separated and the aqueous layer was extracted with ethyl acetate. The combined organic layers were evaporated and the crude product was purified by flash chromatography with a 20-60% ethyl acetate in hexanes gradient to yield 120 mg of 5-(4′,5′-difluoro-2′-methoxy-biphenyl-4-yloxymethyl)-benzo[d]isoxazol-3-ylamine as a white solid. LC-MS (ES) calculated for C21H16F2N2O3, 382.37. found m/z 383 [M+H]⁺.

### Example 2

### ([5-(4′,5′-Difluoro-2′-methoxy-biphenyl-4-yloxymethyl)-benzo[d] isoxazol-3-yl]-methyl-amine

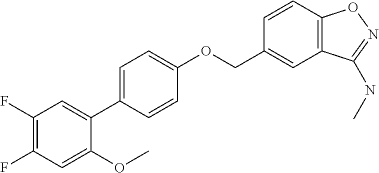

N,N-di-Boc-5-(4′,5′-difluoro-2′-methoxy-biphenyl-4-yloxymethyl)-benzo[d]isoxazol-3-ylamine (0.2 g, 0.343 mmol) and hydrazine hydrate (20 mg, 0.411 mmol) in 5 mL EtOH were stirred for 3.5 hrs and then evaporated. The crude product was purified by flash chromatography with a 0-20% ethyl acetate in hexanes gradient to yield 151 mg of N-Boc-5-(4′,5′-difluoro-2′-methoxy-biphenyl-4-yloxymethyl)-benzo[d]isoxazol-3-ylamine. LC-MS (ES) calculated for C22H18F2N2O3, 396.40. found m/z 397 [M+H]⁺.

N-Boc-5-(4′,5′-difluoro-2′-methoxy-biphenyl-4-yloxymethyl)-benzo[d]isoxazol-3-ylamine (0.24 g, 0.497 mmol), potassium carbonate (0.17 g, 1.23 mmol) and dimethyl-sulfate (57 uL, 0.59 mmol) was stirred for 6 hrs. The solvent was evaporated and the residue was treated with 5 mL of 30% TFA/CH2Cl2 for 1 hr. The crude product was purified by flash chromatography with a 0-40% ethyl acetate in hexanes gradient to yield 180 mg of [5-(4′,5′-difluoro-2′-methoxy-biphenyl-4-yloxymethyl)-benzo[d]isoxazol-3-yl]-methyl-amine as a white solid. LC-MS (ES) calculated for C26H24F2N2O5, 482.49. found m/z 483 [M+H]⁺.

### Example 3

### {[5-(4′,5′-Difluoro-2-methoxy-biphenyl-4-yloxymethyl)-benzo[d] isoxazol-3-yl]-methyl-amino}-acetic acid

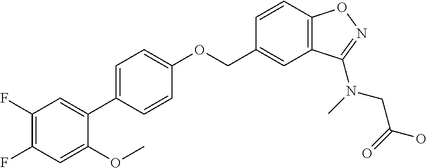

[5-(4′,5′-Difluoro-2′-methoxy-biphenyl-4-yloxymethyl)-benzo[d]isoxazol-3-yl]-methyl-amine (0.15 g, 0.378 mmol), cesium carbonate (63 mg, 0.378 mmol) and ethyl bromoacetate (80 uL, 0.378 mmol) in 5 mL DMF were heated at 120° C. for 20 hrs. Additional cesium carbonate (120 mg, 0.756 mmol) and ethyl bromoacetate (160 uL, 0.756 mmol) were added with heating for 8 hrs. Additional cesium carbonate and ethyl bromoacetate were added two more times with ˜2 days of heating. The reaction was cooled and following an ethyl acetate/H₂O workup, the crude product was purified by flash chromatography with a 0-25% ethyl acetate in hexanes gradient to yield 28 mg of {[5-(4′,5′-difluoro-2′-methoxy-biphenyl-4-yloxymethyl)-benzo[d]isoxazol-3-yl]-methyl-amino}-acetic acid ethyl ester as a viscous oil.

This material was treated LiOH.H₂O (5 mg, 0.116 mmol) in 6 mL of THF/H₂O for 4 hr. Following an ethyl acetate/H₂O workup, the crude product was purified by trituration with CH₂Cl₂ and pentane (1/5) to yield 20 mg of {[5-(4′,5′-difluoro-2′-methoxy-biphenyl-4-yloxymethyl)-benzo[d]isoxazol-3-yl]-methyl-amino}-acetic acid as a white solid. LC-MS (ES) calculated for C24H20F2N2O5, 454.43. found m/z 455 [M+H]⁺.

### Example 4

### 5-(4′,5′-Difluoro-2′-methoxy-biphenyl-4-yloxymethyl)-benzo[d]isoxazol-3-ol

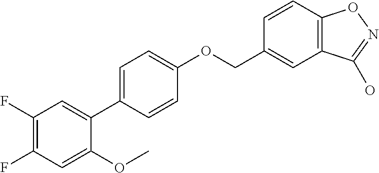

Similar to a procedure in WO2005089753, 5-methyl-salicylic acid methyl ester (10 g, 60 mmol) and 50% hydroxylamine/H₂O (25 mL, 380 mmol) in 25 mL H₂O and 90 mL dioxane was stirred at RT for 4.5 hrs. Workup yielded 10 g of 2,N-dihydroxy-5-methyl-benzamide as an off-white solid.

The crude 2,N-dihydroxy-5-methyl-benzamide (3 g, 17.9 mmol) and carbonyl-diimidazole (5.8 g, 35.7 mmol) were refluxed in 500 mL THF for 2 hrs and then evaporated. Water was added to the residue and carefully acidified to pH 1 with 1N HCl. A solid precipitate formed which was filtered, washed with H₂O and dried under high vacuum with heat (65-70° C.) to yield 2.1 g of 5-methyl-benzo[d]isoxazol-3-ol as a white solid.

5-Methyl-benzo[d]isoxazol-3-ol (0.5 g, 3.35 mmol), tetrahydropyran (0.34 g, 4.0 mmol) and p-toluenesulfonic acid/pyridine (“catalytic”) in 10 mL THF were heated to 75° C. in a sealed vessel for 2 hrs. The solvent was evaporated and the crude product was purified by flash chromatography with a 0-30% ethyl acetate in hexanes gradient to yield 520 mg of 5-methyl-3-(tetrahydro-pyran-2-yloxy)-benzo[d]isoxazole as a white solid.

5-Methyl-3-(tetrahydro-pyran-2-yloxy)-benzo[d]isoxazole (0.14 g, 0.6 mmol), N-bromo-succinimide (0.11 g, 0.6 mmol) and 2,2′-azobis(2-methylpropionitrile) (10 mg, 0.06 mmol) in 10 mL CCl₄ were stirred for 3.5 hrs. The reaction was concentrated and the crude product was purified by flash chromatography with 0-30% ethyl acetate in hexanes to yield 55 mg of 5-bromomethyl-3-(tetrahydro-pyran-2-yloxy)-benzo[d]isoxazole as a 3:1 mixture with starting material.

This semi-pure product (55 mg, 0.176 mmol), 3-(4′,5′-difluoro-2′-methoxy-biphenyl-4-ol (42 mg, 0.176 mmol) and potassium carbonate (24 mg, 0.176 mmol) in 5 mL DMF was stirred overnight. After an aqueous/ethyl acetate workup, the crude product was purified by flash chromatography with a 0-60% ethyl acetate in hexanes gradient to yield 55 mg of 5-(4′,5′-difluoro-2′-methoxy-biphenyl-4-yloxymethyl)-3-(tetrahydro-pyran-2-yloxy)benzo[d]isoxazole.

This material in 5 mL MeOH was treated with 1N HCl (240 uL, 0.236 mmol) for 40 min followed by heating to 75° C. for 30 min. Following an ethyl acetate/H₂O workup, the crude product was purified by flash chromatography with a 0-2% MeOH in CH₂Cl₂ gradient and precipitation from CH₂Cl₂ with hexanes (1:5) to yield 31 mg of 5-(4′,5′-difluoro-2′-methoxy-biphenyl-4-yloxymethyl)-benzo[d]isoxazol-3-ol as a white solid. LC-MS (ES) calculated for C21H15F2NO4, 383.35. found m/z 384.1 [M+H]⁺.

### Example 5

### [5-(4′,5′-Difluoro-2′-methoxy-biphenyl-4-yloxymethyl)-benzo[d]isoxazol-3-yloxy]-acetic acid

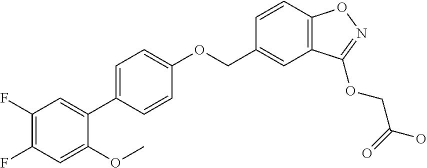

5-(4′,5′-Difluoro-2′-methoxy-biphenyl-4-yloxymethyl)-benzo[d]isoxazol-3-ol (70 mg, 0.183 mmol), cesium carbonate (46 mg, 0.275 mmol) and ethyl bromoacetate (89 mg, 0.275 mmol) in 5 mL DMF were stirred at RT overnight. The solvent was removed in vacuo and the crude product was purified by flash chromatography with a 0-40% ethyl acetate in hexanes gradient to yield 51 mg of [5-(4′,5′-difluoro-2′-methoxy-biphenyl-4-yloxymethyl)-benzo[d]isoxazol-3-yloxy]-acetic acid ethyl ester as a colorless, viscous oil.

This material was treated with LiOH.H₂O in 6 mL THF/H₂O (5:1) at RT for 2 hrs. After an 1 N aqueous HCl/ethyl acetate workup, the crude product was triturated with CH₂Cl₂/hexanes to yield 43 mg of [5-(4′,5′-difluoro-2′-methoxy-biphenyl-4-yloxymethyl)-benzo[d]isoxazol-3-yloxy]-acetic acid as a white solid. LC-MS (ES) calculated for C23H17F2NO6, 441.39. found m/z 442 [M+H]⁺.

### Example 6

### 2-[5-(4′,5′-Difluoro-2′-methoxy-biphenyl-4-yloxymethyl)-benzo[d]isoxazol-3-yloxy]-2-methyl-propionic acid

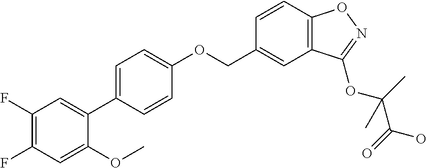

5-(4′,5′-Difluoro-2′-methoxy-biphenyl-4-yloxymethyl)-benzo[d]isoxazol-3-ol (70 mg, 0.183 mmol), cesium carbonate (46 mg, 0.275 mmol) and ethyl 2-bromoisobutyrate (54 mg, 0.275 mmol) were reacted as above to yield 56 mg of 2-[5-(4′,5′-difluoro-2′-methoxy-biphenyl-4-yloxymethyl)-benzo[d]isoxazol-3-yloxy]-2-methyl-propionic acid as a white solid. LC-MS (ES) calculated for C25H21F2NO6, 469.45. found m/z 470 [M+H]⁺.

### Example 7

### 2-[5-(4′,5′-Difluoro-2′-methoxy-biphenyl-4-yloxymethyl)-benzo[d] isoxazol-3-yloxy]-propionic acid

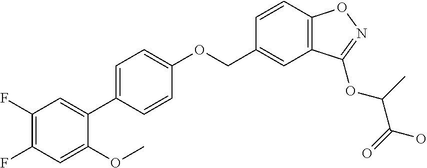

5-(4′,5′-Difluoro-2′-methoxy-biphenyl-4-yloxymethyl)-benzo[d]isoxazol-3-ol (70 mg, 0.183 mmol), cesium carbonate (46 mg, 0.275 mmol) and ethyl 2-bromopropionate (50 mg, 0.275 mmol) were reacted as above to yield 49 mg of 2-[5-(4′,5′-difluoro-2′-methoxy-biphenyl-4-yloxymethyl)-benzo[d]isoxazol-3-yloxy]-propionic acid as a white solid. LC-MS (ES) calculated for C24H19F2NO6, 455.42. found m/z 456 [M+H]⁺.

### Example 8

### [5-(4′,5′-Difluoro-2′-methoxy-biphenyl-4-yloxymethyl)-benzo[d] isoxazol-3-yloxy]-phenyl-acetic acid

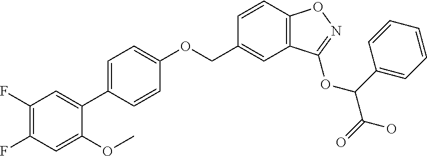

5-(4′,5′-Difluoro-2′-methoxy-biphenyl-4-yloxymethyl)-benzo[d]isoxazol-3-ol (70 mg, 0.183 mmol), cesium carbonate (46 mg, 0.275 mmol) and bromo-phenyl-acetic acid ethyl ester (67 mg, 0.275 mmol) were reacted as above to yield 65 mg of [5-(4′,5′-difluoro-2′-methoxy-biphenyl-4-yloxymethyl)-benzo[d]isoxazol-3-yloxy]-phenyl-acetic acid as a white solid. LC-MS (ES) calculated for C29H21F2NO6, 517.49. found m/z 518 [M+H]⁺.

### Example 9

### 3-[5-(4′,5′-Difluoro-2′-methoxy-biphenyl-4-yloxymethyl)-benzo[d]isoxazol-3-yloxy]-propionic acid

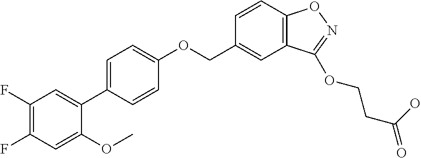

5-(4′,5′-Difluoro-2′-methoxy-biphenyl-4-yloxymethyl)-benzo[d]isoxazol-3-ol (70 mg, 0.183 mmol), DEAD (47 mg, 0.275 mmol), triphenylphosphine (72 mg, 0.275 mol) and 3-hydroxypropionate t-butyl ester (40 mg, 0.275 mmol) in 5 mL THF were stirred overnight. Additional DEAD (47 mg, 0.275 mmol), triphenylphosphine (72 mg, 0.275 mol) and 3-hydroxypropionate t-butyl ester (40 mg, 0.275 mmol) were added and stirred overnight. The solvent was removed in vacuo and the crude product was purified by flash chromatography with a 0-30% ethyl acetate in hexanes gradient to yield 71 mg of 3-[5-(4′,5′-difluoro-2′-methoxy-biphenyl-4-yloxymethyl)-benzo[d]isoxazol-3-yloxy]-propionic acid tert-butyl ester. LC-MS (ES) calculated for C28H27F2NO6, 511.53. found m/z 512 [M+H]⁺.

The above ester was treated with 5 mL 30% TFA/CH₂Cl₂ for 1 hr. Following evaporation, the crude product was precipitated from CH₂Cl₂ with pentane to yield 51 mg of 3-[5-(4′,5′-difluoro-2′-methoxy-biphenyl-4-yloxymethyl)-benzo[d]isoxazol-3-yloxy]-propionic acid as a white solid. LC-MS (ES) calculated for C24H19F2NO6, 455.42. found m/z 456 [M+H]⁺.

### Example 10

### 7-(2′,4′,5′-Trifluoro-biphenyl-4-yl-oxymethyl)-benzo[d]isoxazol-3-ol

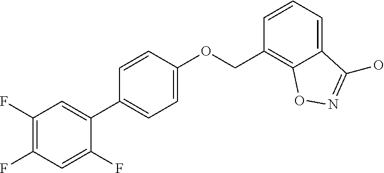

7-Methyl-benzo[d]isoxazol-3-ol (890 mg, 5.967 mmol) was suspended in 20 ml of methylene chloride. To this suspension were added trityl chloride (1.99 g, 7.18 mmol) and pyridine (472 mg, 5.967 mmol). The mixture was refluxed for 1 hr, cooled and diluted with methylene chloride and water. The organic layer was washed with brine, dried and concentrated in vacuo. The residue was treated with methylene chloride and ethyl acetate and filtered. The solution was concentrated in vacuo to give the crude product which was purified by using an ISCO (120 g) column chromatography, eluting with 2-30% ethyl acetate in hexanes to obtain 7-methyl-3-trityloxy-benzo[d]isoxazole as a white solid (1.73 g, 74.1%). 1H NMR (DMSO-d₆) δ ppm: 7.47 (d, J=7.5 Hz, 7H), 7.39 (d, J=7.8 Hz, 1H), 7.27-7.36 (t, J=7.5 Hz, 6H), 7.19-7.27 (m, 3H), 7.11 (t, J=7.8 Hz, 1H), 2.15 (s, 3H). HR-MS calculated for C27H21NO2 (m/e) 414.1464. found m/z 414.1462 [M+Na].

7-Methyl-3-trityloxy-benzo[d]isoxazole (1.72 g, 4.394 mmol) was suspended in 22 ml of methylene chloride. To this suspension were added NBS (869 mg, 4.833 mmol) and V65 (100 mg, 0.404 mmol). The mixture was refluxed for 36 hrs, cooled and diluted with methylene chloride and water. The organic layer was separated and washed with brine, dried and solvents were evaporated. The crude product was purified using an ISCO (120 g) column chromatography, eluting with 2-15% ethyl acetate in hexanes to obtain 7-bromomethyl-3-trityloxy-benzo[d]isoxazole as a white solid (1.11 g, 53.7%). 1H NMR (DMSO-d₆) δ ppm: 7.74 (d, J=7.2 Hz, 1H), 7.56 (d, J=7.2 Hz, 1H), 7.50 (d, J=7.2 Hz, 6H), 7.15-7.37 (m, 10H), 4.60 (s, 2H). HR-MS calculated for C27H20BrNO2, 492.0569. found m/z 492.0570 [M+Na].

2′,4′,5′-Trifluoro-biphenyl-4-ol (250 mg, 1.11 mmol), 7-bromomethyl-3-trityloxy-benzo[d]isoxazole (550.7 mg 1.17 mmol) and potassium carbonate (308.2 mg, 2.23 mmol) were suspended in 15 ml of acetone. The reaction was refluxed for 12 hrs. The mixture was diluted with ethyl acetate and water. The organic layer was washed with brine, dried and solvents were evaporated. The crude product was purified using an ISCO (80 g) column chromatography, eluting with 2-25% ethyl acetate in hexanes to obtain 7-(2′,4′,5′-trifluoro-biphenyl-4-yl-oxymethyl)-3-trityloxy-benzo[d]isoxazole as a white solid (680 mg, 99.4%). 1H NMR (DMSO-d₆) δ ppm: 7.78 (d, J=6.9 Hz, 1H), 7.57 (d, J=7.2 Hz, 1H), 7.44 (d, J=7.2 Hz, 6H), 7.17-7.40 (m, 14H), 6.86 (d, J=8.8 Hz, 2H), 5.16 (s, 2H), 3.76 (s, 3H) HR-MS calculated for C39H26F3NO3, 636.1757. found m/z 636.1759 [M+Na].

7-(2′,4′,5′-Trifluoro-biphenyl-4-yl oxymethyl)-3-trityloxy-benzo[d]isoxazole (677 mg, 1.10 mmol) was suspended in 3 ml of THF and 15 ml of methanol. Aqueous HCl solution (1.0N, 12 ml) was added and the resulted suspension was refluxed for 1 hr. Additional 6 ml of THF was added and the clear solution was refluxed 1 hr further. The mixture was then concentrated and then diluted with ethyl acetate and water. The organic layer was washed with brine, dried and solvents were evaporated. The solid was treated methylene chloride/ether and hexane to afford 7-(2′,4′,5′-trifluoro-biphenyl-4-yl-oxymethyl)-benzo[d]isoxazol-3-ol as a white solid (350 mg, 85.4%). 1H NMR (DMSO-d₆) δ ppm: 12.47 (br. S., 1H), 7.58-7.79 (m, 4H), 7.51 (d, J=8.8 Hz, 2H), 7.36 (t, J=7.5 Hz, 1H), 7.17 (d, J=8.8 Hz, 2H), 5,42 (s, 2H). HR-MS calculated for C20H12F3NO3, 372.0842. found m/z 372.0843 [M+H].

### Example 11

### 7-(4′,5′-Difluoro-2′-methoxy-biphenyl-4-yloxymethyl)-benzo[d] isoxazol-3-ol

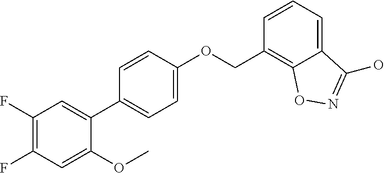

4′,5′-Difluoro-2′-methoxy-biphenyl-4-ol (265 mg, 1.12 mmol), 7-bromomethyl-3-trityloxy-benzo[d]isoxazole (554.1 mg, 1.18 mmol) and potassium carbonate (310 mg, 2.244 mmol) were suspended in 15 ml of acetone. The reaction was refluxed for 12 hrs. The mixture was diluted with ethyl acetate and water. The organic layer was washed with brine, dried and solvents were evaporated. The crude product was purified by using an ISCO (80 g) column chromatography, eluting with 2-25% ethyl acetate in hexanes to obtain 7-(4′,5′-difluoro-2′-methoxy-biphenyl-4-yloxymethyl)-3-trityloxy-benzo[d]isoxazole as a white solid (553 mg, 78.8%). 1H NMR (DMSO-d₆) δ ppm: 7.78 (d, J=6.9 Hz, 1H), 7.57 (d, J=7.2 Hz, 1H), 7.44 (d, J=7.2 Hz, 6H), 7.17-7.40 (m, 14H), 6.86 (d, J=8.8 Hz, 2H), 5.16 (s, 2H), 3.76 (s, 3H). HR-MS calculated for C40H29F2NO4, 648.1957. found m/z 648.1957 [M+Na].

7-(4′,5′-Difluoro-2′-methoxy-biphenyl-4-yloxymethyl)-3-trityloxy-benzo[d]isoxazole (543 mg, 0.87 mmol) was suspended in 2.5 ml of THF and 10 ml of methanol. 1.0N HCl (9.5 ml) was added and the resulted suspension was refluxed for 1 hr. Additional 6 ml of THF was added and the clear solution was refluxed 1 hr further. The mixture was then concentrated and then diluted with ethyl acetate and water. The organic layer was washed with brine, dried and solvents were evaporated. The solid was treated with methylene chloride/ether and hexane to afford 7-(4′,5′-difluoro-2′-methoxy-biphenyl-4-yloxymethyl)-benzo[d]isoxazol-3-ol as a white solid (300 mg, 90.2%). 1H NMR (DMSO-d₆) δ ppm: 12.47 (br. S., 1H), 7.75 (d, J=8.5 Hz, 1H), 7.72 (d, J=6.9 Hz, 1H), 7.42 (d, J=8.6 Hz, 2H), 7.31-7.40 (m, 2H), 7.23 (dd, J=13.0, 7.2 Hz, 1H), 7.09 (d, J=8.6 Hz, 2H), 5.39 (s, 2H), 3.76 (s, 3H). HR-MS calculated for C20H12F3NO3; 384.1042. found m/z 384.1041 [M+H].

### Example 12

### 7-(2′-Chloro-4′,5′-difluoro-biphenyl-4-yloxymethyl)-benzo[d]isoxazol-3-ol

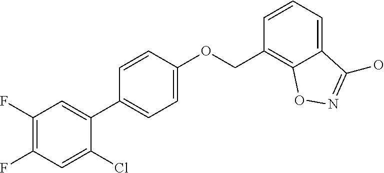

2′-Chloro-4′,5′-difluoro-biphenyl-4-ol (205 mg, 0.85 mmol), 7-bromomethyl-3-trityloxy-benzo[d]isoxazole (480.9 mg, 1.02 mmol) and 1.2 equivalents of potassium carbonate (141.3 mg, 1.022 mmol) were suspended in 10 ml of DMF. The reaction was stirred at room temperature overnight. The mixture was diluted with ethyl acetate and water. The organic layer was washed with brine, dried and solvents were evaporated. The crude product was purified by using an ISCO (80 g) column chromatography, eluting with 2-25% ethyl acetate in hexanes to obtain 7-(2′-chloro-4′,5′-difluoro-biphenyl-4-yloxymethyl)-3-trityloxy-benzo[d]isoxazole as a white solid (424 mg, 79.0%). ¹H NMR 300 MHz (DMSO-d₆) δ ppm: 7.76-7.87 (m, 2H), 7.48-7-61 (m, 2H), 7.45 (d, J=7.2 Hz, 6H), 7.17-7.35 (m, 12H), 6.93 (d, J=8.8 Hz, 2H), 5.18 (s, 2H). HR-MS calculated for C39H26ClF2NO3, 652.1461. found m/z 652.1458 [M+Na].

7-(2′-Chloro-4′,5′-difluoro-biphenyl-4-yloxymethyl)-3-trityloxy-benzo[d]isoxazole (410 mg, 0.65 mmol) was suspended in 2.0 ml of THF and 9 ml of methanol. HCl solution (1.0N, 6.5 ml) was added and the resulted suspension was adding additional 3 ml of THF to obtain clear solution which was refluxed for 2 hrs. The mixture was then concentrated and diluted with ethyl acetate and water. The organic layer was washed with brine, dried and solvents were evaporated. The crude product was purified using an ISCO (50 g) column chromatography, eluting with 20-60% ethyl acetate in hexanes to obtain 7-(2′-chloro-4′,5′-difluoro-biphenyl-4-yloxymethyl)-benzo[d]isoxazol-3-ol as a white solid (85 mg, 33.7%). HR-MS calculated for C20H12ClF2NO3, 386.0401. found m/z 386.0402 [M−H].

### Example 13

### 7-(4′,5′-Difluoro-2′-methyl-biphenyl-4-yloxymethyl)-benzo[d]isoxazol-3-ol

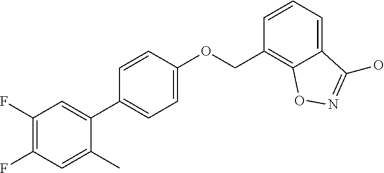

4′,5′-Difluoro-2′-methyl-biphenyl-4-ol (200 mg, 0.91 mmol), 7-bromomethyl-3-trityloxy-benzo[d]isoxazole (469.8 mg, 1.0 mmol) and potassium carbonate (150.6 mg, 1.09 mmol) were suspended in 10 ml of DMF. The reaction was stirred at room temperature overnight. The mixture was diluted with ethyl acetate and water. The organic layer was washed with brine, dried and solvents were evaporated. The crude product was purified using an ISCO (40 g) column chromatography, eluting with 2-25% ethyl acetate in hexanes to obtain 7-(4′,5′-difluoro-2′-methyl-biphenyl-4-yloxymethyl)-3-trityloxy-benzo[d]isoxazole as a foam solid (486 mg, 87.8. %). 1H NMR (DMSO-d₆) δ ppm: 7.79 (d, J=7.2 Hz, 1H), 7.58 (d, J=7.8 Hz, 1H), 7.44 (d, J=7.5 Hz, 6H), 7.38 (dd, J=12.1, 8.5 Hz, 1H), 7.15-7.31 (m, 13H), 6.90 (d, J=8.5 Hz, 2H), 5.16 (s, 2H), 2.19 (s, 3H). HR-MS calculated for C40H29F2NO3, 632.2008. found m/z 632.2008 [M+Na].

7-(4′,5′-Difluoro-2′-methyl-biphenyl-4-yloxymethyl)-3-trityloxy-benzo[d]isoxazole (480 mg, 0.79 mmol) was suspended in 5.0 ml of THF and 10 ml of methanol. 1.0N HCl (10.0 ml) and the an additional 3 ml of THF were added to obtain a clear solution, which was refluxed for 2 hrs. The mixture was then concentrated and diluted with ethyl acetate and water. The organic layer was washed with brine, dried and solvents were evaporated. The crude product was treated with methylene chloride, ether and hexane and filtered to obtain 7-(4′,5′-difluoro-2′-methyl-biphenyl-4-yloxymethyl)-benzo[d]isoxazol-3-ol as a white solid (174 mg, 60.2%). 1H NMR (DMSO-d₆) δ ppm: 12.46 (s, 1H), 7.75 (t, J=7.2 Hz, 2H), 7.33-7.44 (m, 2H), 7.30 (d, J=8.8 Hz, 2H), 7.19-7.28 (m, 1H), 7.13 (d, J=8.8 Hz, 2H), 5.39 (s, 2H), 2.19 (s, 3H). HR-MS calculated for C21H15F2NO3, 368.1093. found m/z 368.1093 [M+H].

### Example 14

### [[5-(4′,5′-Difluoro-2′-methoxy-biphenyl-4-yloxymethyl)-benzo[d] isoxazol-3-yl]-(2-methoxy-ethyl)-amino]-acetic acid

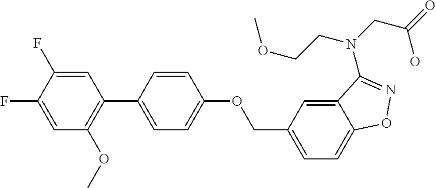

To [5-(4′,5′-difluoro-2′-methoxy-biphenyl-4-yloxymethyl)-benzo[d]isoxazol-3-yl]-carbamic acid tert-butyl ester (100 mg, 0.21 mmol, 1 eq) was added dimethylformamide (1 ml) and then lithium bis(trimethylsilyl)amide (1 M in THF, 0.25 ml, 0.25 mmol, 1.2 eq). The reaction stirred for 5 min. and then 1-bromo-2-methoxy-ethane (0.039 ml, 0.41 mmol, 2 eq) was added. The reactions was placed under nitrogen, sealed, and heated to 80° C. for overnight (˜17 hr). To the reaction was added more 1-bromo-2-methoxy-ethane (0.020 ml) and heated to 80° C. for 4.5 hr. To the reaction was added more lithium bis(trimethylsilyl)amide (1 M in THF, 0.10 ml), stirred for 10 min and then 1-bromo-2-methoxy-ethane (0.040 ml) was added. The reaction was heated to 80° C. for 5 hr and allowed to cool to room temperature overnight. The reaction was diluted with dimethylsulfoxide (1 ml) and purified by HPLC with a 50-100% acetonitrile in water gradient and dried from dichloromethane/hexanes mixture to yield [5-(4′,5′-difluoro-2′-methoxy-biphenyl-4-yloxymethyl)-benzo[d]isoxazol-3-yl]-(2-methoxy-ethyl)-carbamic acid tert-butyl ester as a clear film (60.5 mg, 54% yield; mixture of product and product without tert-butyl carbamate ester, 87/33% respectively by UV). LC-MS (ES) calculated for C29H30F2N2O6, 540.2. found m/z 541 [M+H]⁺.

To [5-(4′,5′-difluoro-2′-methoxy-biphenyl-4-yloxymethyl)-benzo[d]isoxazol-3-yl]-(2-methoxy-ethyl)-carbamic acid tert-butyl ester was added dichloromethane (3 ml) and trifluoroacetic acid (1 ml). The reaction was stirred at room temperature for 2.5 hr and then diluted with ethyl acetate (50 ml). The organic layer was washed two times with aqueous sodium bicarbonate (25 ml of saturated solution/25 ml of water), brine (saturated NaCl), dried over magnesium sulfate, filtered, and concentrated under reduced pressure. The material was dried from dichloromethane/hexanes mixtures to yield [5-(4′,5′-difluoro-2′-methoxy-biphenyl-4-yloxymethyl)-benzo[d]isoxazol-3-yl]-(2-methoxy-ethyl)-amine as an opaque film (46 mg, 93% yield). LC-MS (ES) calculated for C24H22F2N2O4, 440.2. found m/z 441 [M+H]⁺.

To a vial (8 ml) containing [5-(4′,5′-difluoro-2′-methoxy-biphenyl-4-yloxymethyl)-benzo[d]isoxazol-3-yl]-(2-methoxy-ethyl)-amine (44 mg, 0.10 mmol, 1 eq) was added dimethylformamide (0.2 ml), lithium bis(trimethylsilyl)amide (1 M in THF, 0.12 ml, 0.12 mmol, 1.2 eq), and ethyl bromo acetate (0.045 ml, 0.40 mmol, 4 eq). The reaction was placed under nitrogen, sealed, and heated to 100° C. for 4.5 hr. An additional 1.2 equivalents of lithium bis(trimethylsilyl)amide was added, the reaction heated to 100° C. for 7 hr, and allowed to warm to room temperature over night. The reaction was partitioned between ethyl acetate (4 mL) and water (4 ml), the organic layer separated, and the aqueous layer extracted with ethyl acetate (4 mL). The organic layers were washed with brine (saturated NaCl), combined, dried over magnesium sulfate, filtered, evaporated under reduced pressure, dissolved in minimal dichloromethane and purified by flash chromatography with a 0-40% ethyl acetate in hexanes gradient to yield [[5-(4′,5′-difluoro-2′-methoxy-biphenyl-4-yloxymethyl)-benzo[d]isoxazol-3-yl]-(2-methoxy-ethyl)-amino]-acetic acid ethyl ester as a clear film (18 mg, 34% yield). LC-MS (ES) calculated for C28H28F2N2O6, 526.2. found m/z 527 [M+H]⁺.

To [[5-(4′,5′-difluoro-2′-methoxy-biphenyl-4-yloxymethyl)-benzo[d]isoxazol-3-yl]-(2-methoxy-ethyl)-amino]-acetic acid ethyl ester (18 mg, 0.034 mmol, 1 eq.) was added THF (0.5 mL), water (0.5 mL) and LiOH—H₂O (4 mg, 0.1 mmol, 3 eq.) and the reaction allowed to stir at room temperature over night (17 hr). The reaction was partitioned between ethyl acetate (25 mL) and aqueous HCl (0.1 M, 25 mL), the organic layer separated, and the aqueous layer extracted with ethyl acetate (25 mL). The organic layers were washed with brine (saturated NaCl), combined, dried over magnesium sulfate, filtered, evaporated under reduced pressure, resuspended in acetonitrile/water mixture and dried by lyophilization to yield [[5-(4′,5′-difluoro-2′-methoxy-biphenyl-4-yloxymethyl)-benzo[d]isoxazol-3-yl]-(2-methoxy-ethyl)-amino]-acetic acid as a white solid (18 mg, 100% yield). LC-MS (ES) calculated for C26H24F2N2O6, 498.2. found m/z 499 [M+H]⁺.

### Example 15

### [[5-(4′,5′-Difluoro-2′-methoxy-biphenyl-4-yloxymethyl)-benzo[d] isoxazol-3-yl]-(3-methoxy-propyl)-amino]-acetic acid

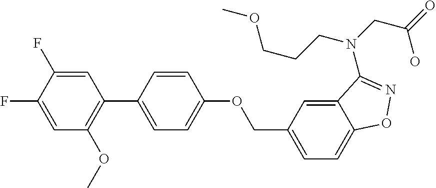

[5-(4′,5′-Difluoro-2′-methoxy-biphenyl-4-yloxymethyl)-benzo[d]isoxazol-3-yl]-(3-methoxy-propyl)-carbamic acid tert-butyl ester was synthesized by a procedure similar to [5-(4′,5′-difluoro-2′-methoxy-biphenyl-4-yloxymethyl)-benzo[d]isoxazol-3-yl]-(2-methoxy-ethyl)-carbamic acid tert-butyl ester from starting materials [5-(4′,5′-difluoro-2′-methoxy-biphenyl-4-yloxymethyl)-benzo[d]isoxazol-3-yl]-carbamic acid tert-butyl ester and 1-bromo-3-methoxy-propane to yield the product as a film (106 mg, 93% yield). LC-MS (ES) calculated for C30H32F2N2O6, 554.2. found m/z 555 [M+H]⁺.

[5-(4′,5′-Difluoro-2′-methoxy-biphenyl-4-yloxymethyl)-benzo[d] isoxazol-3-yl]-(3-methoxy-propyl)-amine was synthesized by a procedure similar to [5-(4′,5′-difluoro-2′-methoxy-biphenyl-4-yloxymethyl)-benzo[d]isoxazol-3-yl]-(2-methoxy-ethyl)-amine from starting material [5-(4′,5′-difluoro-2′-methoxy-biphenyl-4-yloxymethyl)-benzo[d] isoxazol-3-yl]-(3-methoxy-propyl)-carbamic acid tert-butyl ester to yield the product as a film (79 mg, 90% yield). LC-MS (ES) calculated for C25H24F2N2O4, 454.2. found m/z 455 [M+H]⁺.

[[5-(4′,5′-Difluoro-2′-methoxy-biphenyl-4-yloxymethyl)-benzo[d]isoxazol-3-yl]-(3-methoxy-propyl)-amino]-acetic acid ethyl ester was synthesized by a procedure similar to [[5-(4′,5′-difluoro-2′-methoxy-biphenyl-4-yloxymethyl)-benzo[d]isoxazol-3-yl]-(2-methoxy-ethyl)-amino]-acetic acid ethyl ester from starting materials [5-(4′,5′-difluoro-2′-methoxy-biphenyl-4-yloxymethyl)-benzo[d]isoxazol-3-yl]-(3-methoxy-propyl)-amine and ethyl bromo acetate to yield the product as a white solid (26 mg, 28% yield). LC-MS (ES) calculated for C29H30F2N2O6, 540.2. found m/z 541 [M+H]⁺.

[[5-(4′,5′-Difluoro-2′-methoxy-biphenyl-4-yloxymethyl)-benzo[d]isoxazol-3-yl]-(3-methoxy-propyl)-amino]-acetic acid was synthesized by a procedure similar to [[5-(4′,5′-difluoro-2′-methoxy-biphenyl-4-yloxymethyl)-benzo[d]isoxazol-3-yl]-(2-methoxy-ethyl)-amino]-acetic acid from starting material [[5-(4′,5′-difluoro-2′-methoxy-biphenyl-4-yloxymethyl)-benzo[d]isoxazol-3-yl]-(3-methoxy-propyl)-amino]-acetic acid ethyl ester to yield the product as a white solid (19 mg, 77% yield). LC-MS (ES) calculated for C27H26F2N2O6, 512.2. found m/z 513 [M+H]⁺.

### Example 16

### {[5-(4′,5′-Difluoro-2′-methoxy-biphenyl-4-yloxymethyl)-benzo[d]isoxazol-3-yl]-ethyl-amino}-acetic acid

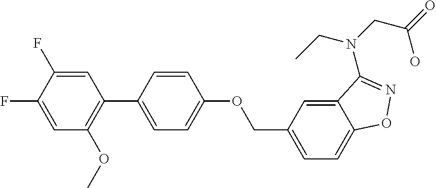

[5-(4′,5′-Difluoro-2′-methoxy-biphenyl-4-yloxymethyl)-benzo[d]isoxazol-3-yl]-ethyl-carbamic acid tert-butyl ester was synthesized by a procedure similar to [5-(4′,5′-difluoro-2′-methoxy-biphenyl-4-yloxymethyl)-benzo[d]isoxazol-3-yl]-(2-methoxy-ethyl)-carbamic acid tert-butyl ester from starting materials [5-(4′,5′-difluoro-2′-methoxy-biphenyl-4-yloxymethyl)-benzo[d]isoxazol-3-yl]-carbamic acid tert-butyl ester and ethyl iodide to yield the product as an opaque film (107 mg, 102% yield). LC-MS (ES) calculated for C28H28F2N2O5, 510.2. found m/z 555 [M+H]⁺.

[5-(4′,5′-Difluoro-2′-methoxy-biphenyl-4-yloxymethyl)-benzo[d]isoxazol-3-yl]-ethyl-amine was synthesized by a procedure similar to [5-(4′,5′-difluoro-2′-methoxy-biphenyl-4-yloxymethyl)-benzo[d]isoxazol-3-yl]-(2-methoxy-ethyl)-amine from starting material [5-(4′,5′-difluoro-2′-methoxy-biphenyl-4-yloxymethyl)-benzo[d]isoxazol-3-yl]-ethyl-carbamic acid tert-butyl ester to yield the product was a yellow solid/film (79 mg, 93% yield). LC-MS (ES) calculated for C23H20F2N2O3, 410.1. found m/z 411 [M+H]⁺.

{[5-(4′,5′-Difluoro-2′-methoxy-biphenyl-4-yloxymethyl)-benzo[d]isoxazol-3-yl]-ethyl-amino}-acetic acid ethyl ester was synthesized by a procedure similar to [[5-(4′,5′-difluoro-2′-methoxy-biphenyl-4-yloxymethyl)-benzo[d]isoxazol-3-yl]-(2-methoxy-ethyl)-amino]-acetic acid ethyl ester from starting materials [5-(4′,5′-difluoro-2′-methoxy-biphenyl-4-yloxymethyl)-benzo[d]isoxazol-3-yl]-ethyl-amine and ethyl bromo acetate to yield the product as a white solid (24 mg, 25% yield). LC-MS (ES) calculated for C27H26F2N2O5, 496.2. found m/z 497 [M+H]⁺.

{[5-(4′,5′-Difluoro-2′-methoxy-biphenyl-4-yloxymethyl)-benzo[d]isoxazol-3-yl]-ethyl-amino}-acetic acid was synthesized by a procedure similar to [[5-(4′,5′-difluoro-2′-methoxy-biphenyl-4-yloxymethyl)-benzo[d]isoxazol-3-yl]-(2-methoxy-ethyl)-amino]-acetic acid from starting material {[5-(4′,5′-difluoro-2′-methoxy-biphenyl-4-yloxymethyl)-benzo[d]isoxazol-3-yl]-ethyl-amino}-acetic acid ethyl ester to yield the product as a white solid, 20 mg, 90% yield. LC-MS (ES) calculated for C25H22F2N2O5, 468.1. found m/z 469 [M+H]⁺.

### Example 17

### {Cyclopropylmethyl-[5-((4′,5′-difluoro-2′-methoxy-biphenyl-4-yloxymethyl)-benzo[d]isoxazol-3-yl]amino}-acetic acid

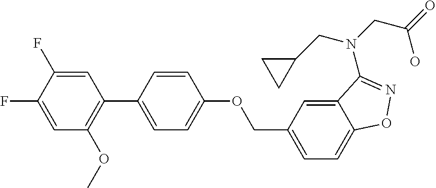

Cyclopropylmethyl-[5-(4′,5′-difluoro-2′-methoxy-biphenyl-4-yloxymethyl)-benzo[d]isoxazol-3-yl]-carbamic acid tert-butyl ester was synthesized by a procedure similar to [5-(4′,5′-difluoro-2′-methoxy-biphenyl-4-yloxymethyl)-benzo[d]isoxazol-3-yl]-(2-methoxy-ethyl)-carbamic acid tert-butyl ester from starting materials [5-(4′,5′-difluoro-2′-methoxy-biphenyl-4-yloxymethyl)-benzo[d]isoxazol-3-yl]-carbamic acid tert-butyl ester and cyclopropyl bromide to yield the product as a yellow clear semi-solid (95 mg, 85% yield). LC-MS (ES) calculated for C30H30F2N2O5, 536.2. found m/z 537 [M+H]⁺.

Cyclopropylmethyl-[5-(4′,5′-difluoro-2′-methoxy-biphenyl-4-yloxymethyl)-benzo[d]isoxazol-3-yl]-amine was synthesized by a procedure similar to [5-(4′,5′-difluoro-2′-methoxy-biphenyl-4-yloxymethyl)-benzo[d]isoxazol-3-yl]-(2-methoxy-ethyl)-amine from starting material cyclopropylmethyl-[5-(4′,5′-difluoro-2′-methoxy-biphenyl-4-yloxymethyl)-benzo[d]isoxazol-3-yl]-carbamic acid tert-butyl ester to yield the product as a yellow solid/film (70 mg, 92% yield). LC-MS (ES) calculated for C25H22F2N2O3, 436.2. found m/z 437 [M+H]⁺.

{Cyclopropylmethyl-[5-(4′,5′-difluoro-2′-methoxy-biphenyl-4-yloxymethyl)-benzo[d]isoxazol-3-yl]-amino}-acetic acid ethyl ester was synthesized by a procedure similar to [[5-(4′,5′-difluoro-2′-methoxy-biphenyl-4-yloxymethyl)-benzo[d]isoxazol-3-yl]-(2-methoxy-ethyl)-amino]-acetic acid ethyl ester from starting materials cyclopropylmethyl-[5-(4′,5′-difluoro-2′-methoxy-biphenyl-4-yloxymethyl)-benzo[d]isoxazol-3-yl]-amine and ethyl bromo acetate to yield the product as a white solid (14 mg, 17% yield). LC-MS (ES) calculated for C29H28F2N2O5, 522.2. found m/z 523 [M+H]⁺.

{Cyclopropylmethyl-[5-(4′,5′-difluoro-2′-methoxy-biphenyl-4-yloxymethyl)-benzo[d]isoxazol-3-yl]-amino}-acetic acid was synthesized by a procedure similar to [[5-(4′,5′-Difluoro-2′-methoxy-biphenyl-4-yloxymethyl)-benzo[d]isoxazol-3-yl]-(2-methoxy-ethyl)-amino]-acetic acid from starting material {cyclopropylmethyl-[5-(4′,5′-difluoro-2′-methoxy-biphenyl-4-yloxymethyl)-benzo[d]isoxazol-3-yl]-amino}-acetic acid ethyl ester to yield the product as a white solid (12 mg, 93% yield). LC-MS (ES) calculated for C27H24F2N2O5, 494.2. found m/z 495 [M+H]⁺.

### Example 18

### [[5-(4′,5′-Difluoro-2′-methoxy-biphenyl-4-yloxymethyl)-benzo[d]isoxazol-3-yl]-(tetrahydro-pyran-4-ylmethyl)-amino]-acetic acid

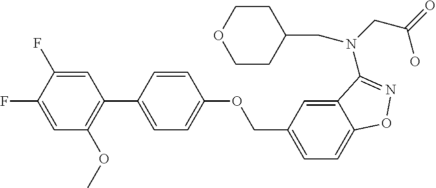

[5-(4′,5′-Difluoro-2′-methoxy-biphenyl-4-yloxymethyl)-benzo[d] isoxazol-3-yl]-(tetrahydro-pyran-4-ylmethyl)-carbamic acid tert-butyl ester was synthesized by a procedure similar to [5-(4′,5′-difluoro-2′-methoxy-biphenyl-4-yloxymethyl)-benzo[d] isoxazol-3-yl]-(2-methoxy-ethyl)-carbamic acid tert-butyl ester from starting materials [5-(4′,5′-difluoro-2′-methoxy-biphenyl-4-yloxymethyl)-benzo[d]isoxazol-3-yl]-carbamic acid tert-butyl ester and tetrahydro-pyran-4-ylmethyl bromide to yield the product as a clear film (72 mg, 60% yield). LC-MS (ES) calculated for C32H34F2N2O6, 580.2. found m/z 581 [M+H]⁺.

[5-(4′,5′-Difluoro-2′-methoxy-biphenyl-4-yloxymethyl)-benzo[d]isoxazol-3-yl]-(tetrahydro-pyran-4-ylmethyl)-amine was synthesized by a procedure similar to [5-(4′,5′-difluoro-2′-methoxy-biphenyl-4-yloxymethyl)-benzo[d]isoxazol-3-yl]-(2-methoxy-ethyl)-amine from starting material [5-(4′,5′-difluoro-2′-methoxy-biphenyl-4-yloxymethyl)-benzo[d]isoxazol-3-yl]-(tetrahydro-pyran-4-ylmethyl)-carbamic acid tert-butyl ester to yield the product as a white solid (50 mg, 83% yield). LC-MS (ES) calculated for C27H26F2N2O4, 480.2. found m/z 481 [M+H]⁺.

[[5-(4′,5′-Difluoro-2′-methoxy-biphenyl-4-yloxymethyl)-benzo[d]isoxazol-3-yl]-(tetrahydro-pyran-4-ylmethyl)-amino]-acetic acid ethyl ester was synthesized by a procedure similar to [[5-(4′,5′-difluoro-2′-methoxy-biphenyl-4-yloxymethyl)-benzo[d]isoxazol-3-yl]-(2-methoxy-ethyl)-amino]-acetic acid ethyl ester from starting materials [5-(4′,5′-difluoro-2′-methoxy-biphenyl-4-yloxymethyl)-benzo[d]isoxazol-3-yl]-(tetrahydro-pyran-4-ylmethyl)-amine and ethyl bromo acetate to yield the product as a clear film (11 mg, 20% yield). LC-MS (ES) calculated for C31H32F2N2O6, 566.2. found m/z 567 [M+H]⁺.

[[5-(4′,5′-Difluoro-2′-methoxy-biphenyl-4-yloxymethyl)-benzo[d]isoxazol-3-yl]-(tetrahydro-pyran-4-ylmethyl)-amino]-acetic acid was synthesized by a procedure similar to [[5-(4′,5′-difluoro-2′-methoxy-biphenyl-4-yloxymethyl)-benzo[d]isoxazol-3-yl]-(2-methoxy-ethyl)-amino]-acetic acid from starting material [[5-(4′,5′-difluoro-2′-methoxy-biphenyl-4-yloxymethyl)-benzo[d]isoxazol-3-yl]-(tetrahydro-pyran-4-ylmethyl)-amino]-acetic acid ethyl ester to yield the product as a white solid (5 mg, 51% yield). LC-MS (ES) calculated for C29H28F2N2O6, 538.2. found m/z 539 [M+H]⁺.

### Example 19

### {[7-(4′,5′-Difluoro-2′-methoxy-biphenyl-4-yloxymethyl)-benzo[d]isoxazol-3-yl]-methyl-amino}-acetic acid

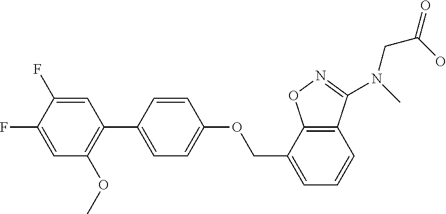

[7-(4′,5′-Difluoro-2′-methoxy-biphenyl-4-yloxymethyl)-benzo[d]isoxazol-3-yl]-methyl-carbamic acid tert-butyl ester was synthesized by a procedure similar to [5-(4′,5′-difluoro-2′-methoxy-biphenyl-4-yloxymethyl)-benzo[d]isoxazol-3-yl]-(2-methoxy-ethyl)-carbamic acid tert-butyl ester from starting materials [7-(4′,5′-difluoro-2′-methoxy-biphenyl-4-yloxymethyl)-benzo[d]isoxazol-3-yl]-carbamic acid tert-butyl ester and methyl iodide to yield the product as an opaque film (107 mg, 102% yield). LC-MS (ES) calculated for C27H26F2N2O5, 496.2. found m/z 497 [M+H]⁺.

[7-(4′,5′-Difluoro-2′-methoxy-biphenyl-4-yloxymethyl)-benzo[d]isoxazol-3-yl]-methyl-amine was synthesized by a procedure similar to [5-(4′,5′-difluoro-2′-methoxy-biphenyl-4-yloxymethyl)-benzo[d]isoxazol-3-yl]-(2-methoxy-ethyl)-amine from starting material [7-(4′,5′-difluoro-2′-methoxy-biphenyl-4-yloxymethyl)-benzo[d]isoxazol-3-yl]-methyl-carbamic acid tert-butyl ester to yield the product as a yellow solid/film (79 mg, 93% yield). LC-MS (ES) calculated for C22H18F2N2O3, 396.1. found m/z 397 [M+H]⁺.

{[7-(4′,5′-Difluoro-2′-methoxy-biphenyl-4-yloxymethyl)-benzo[d]isoxazol-3-yl]-methyl-amino}-acetic acid ethyl ester was synthesized by a procedure similar to [[5-(4′,5′-difluoro-2′-methoxy-biphenyl-4-yloxymethyl)-benzo[d]isoxazol-3-yl]-(2-methoxy-ethyl)-amino]-acetic acid ethyl ester from starting materials [7-(4′,5′-difluoro-2′-methoxy-biphenyl-4-yloxymethyl)-benzo[d]isoxazol-3-yl]-methyl-amine and ethyl bromo acetate to yield the product as a white solid (24 mg, 25% yield). LC-MS (ES) calculated for C26H24F2N2O5, 482.2. found m/z 483 [M+H]⁺.

{[7-(4′,5′-Difluoro-2′-methoxy-biphenyl-4-yloxymethyl)-benzo[d]isoxazol-3-yl]-methyl-amino}-acetic acid was synthesized by a procedure similar to [[5-(4′,5′-difluoro-2′-methoxy-biphenyl-4-yloxymethyl)-benzo[d]isoxazol-3-yl]-(2-methoxy-ethyl)-amino]-acetic acid from starting material {[7-(4′,5′-difluoro-2′-methoxy-biphenyl-4-yloxymethyl)-benzo[d]isoxazol-3-yl]-methyl-amino}-acetic acid ethyl ester to yield the product as a white solid (20 mg, 90% yield). LC-MS (ES) calculated for C24H20F2N2O5, 454.43. found m/z 455 [M+H]⁺.

### Example 20

### 5-(2′,4′,5′-Trifluoro-biphenyl-4-yloxymethyl)-benzo[d]isoxazol-3-ol

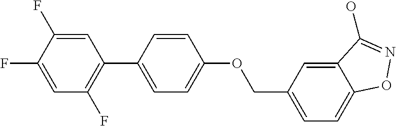

To a solution of 2′,4′,5′-trifluoro-biphenyl-4-ol (1.0 g, 4.46 mmol) in acetone (60 mL) was added 5-bromomethyl-2-trityl-benzo[d]isoxazol-3-one (1.90 g, 4.04 mmol) and potassium carbonate (1.23 g, 8.9 mmol). The mixture was stirred and refluxed for 2 hrs. Solvents were evaporated and the residue was extracted with ethyl acetate and water. The organic layer was washed with brine and dried over sodium sulfate. After the evaporation of solvents, the residue was purified through ISCO flash column chromatography using ethyl acetate and hexanes (100 g silica gel, 0% to 25% linear gradient) to give 5-(2′,4′,5′-trifluoro-biphenyl-4-yloxymethyl)-2-trityl-benzo[d]isoxazol-3-one as a white fluffy material (1.90 g, 77% yield). HRMS-ES(+) cald for C₃₉H₂₆NO₃F₃ [M+Na]⁺ 636.1757. found m/z 636.1753; ¹H-NMR (300 MHz, DMSO-d₆) δ ppm 7.75 (d, J=8.8 Hz, 1H), 7.57-7.71 (m, 3H), 7.38-7.53 (m, 9H), 7.27-7.35 (m, 6H), 7.19-7.27 (m, 3H), 7.09 (d, J=8.8 Hz, 2H), 5.16 (s, 2H).

To a stirred solution of 5-(2′,4′,5′-trifluoro-biphenyl-4-yloxymethyl)-2-trityl-benzo[d]isoxazol-3-one (1.90 g, 3.1 mmol) in THF (60 mL) containing methanol (50 mL) was added aqueous hydrochloric acid (1N, 50 mL) through a dropping funnel over 10 minutes. The mixture was heated to refluxing for 20 minutes and solvents were evaporated. The residue was extracted with ethyl acetate and water. The organic layer was washed with water and dried over sodium sulfate. Solvents were evaporated and the residue was treated with a mixture of ethyl acetate and hexanes (1:4 ratio, 80 mL). The mixture was stirred at room temperature for 5 minutes. The resulting mixture was filtered and washed with a mixture of ethyl acetate in hexanes (1:4 ratio, 20 mL). The white solid was dried to give 5-(2′,4′,5′-trifluoro-biphenyl-4-yloxymethyl)-benzo[d]isoxazol-3-ol (992 mg, 86% yield). HRMS-ES(+) cald for C₂₀H₁₂NO₃F₃ [M+H]⁺ 372.0842. found m/z 372.0840; ¹H-NMR (300 MHz, DMSO-d₆) δ ppm 12.41 (br s, 1H), 7.85 (s, 1H), 7.55-7.74 (m, 4H), 7.50 (d, J=8.7 Hz, 2H), 7.14 (d, J=8.7 Hz, 2H), 5.27 (s, 2H).

### Example 21

### Glycogen Synthase (GS) Assay

The following tests were carried out in order to determine the activity of the compounds of formula (I).

Twelve μl per well of substrate solution containing glycogen (4.32 mg/ml), 2.67 mM UDP-glucose, 21.6 mM phospho(enol)pyruvate and 2.7 mM NADH in 30 mM glycylglycine, pH 7.3 buffer was added into a polystyrene 384-well assay plate (BD Biosciences).

Compound solutions (8 μl/well) at various concentrations (0-300 μM) were added to the assay plate (columns 5-24). Compound solution contains 30 mM glycylglycine, pH 7.3, 40 mM KCl, 20 mM MgCl₂, 9.2% DMSO, with (columns 15-24) or without (columns 5-14) 20 mM glucose 6-phosphate.

Enzyme solution (12 μl/well) containing glycogen synthase (16.88 μg/ml), pyruvate kinase (0.27 mg/ml), lactate dehydrogenase (0.27 mg/ml) in 50 mM Tris-HCl, pH 8.0, 27 mM DTT and bovine serum albumin (BSA, 0.2 mg/ml) was added to the assay plate (columns 3-24). As a blank control, enzyme solution without glycogen synthase was added into the top half wells of columns 1-2. To the bottom half wells of columns 1-2 were added a known activator, glucose 6-phosphate (at final concentration 5 mM) in addition to the enzyme solution. The reaction mixture was incubated at room temperature. The assay plate was then read for absorbance at 340 nm on an Envision reader every 3 minutes up to a total of 15 minutes.

The enzyme activity (with or without compound) was calculated by the reaction rate and represented by the optical density change (μOD) per minute. Percent stimulation of glycogen synthase activity by a compound at various concentrations was calculated by the following formula:

% stimulation=100*Rs/Rt,

wherein Rs is the reaction rate of the enzyme in the presence of compound and Rt is the reaction rate of the enzyme in the absence of compound.

SC₂₀₀ is defined as the compound concentration that is needed to stimulate 200% of the enzyme activity. EC₅₀ is defined as the compound concentration that is needed to give 50% maximum activation.

Compounds from Example 1 through Example 20 were assayed according to assay procedures described above and the results are listed in Table 1 as follows:

TABLE 1
Glycogen Synthase Activation Potency

| Example Number | GS SC₂₀₀ (μM) | GS EC₅₀ (μM) |
|---|---|---|
| 1 | 0.688 | 1.488 |
| 2 | 0.514 | 1.971 |
| 3 | 0.024 | 0.302 |
| 4 | 0.032 | 0.172 |
| 5 | 0.492 | 1.346 |
| 6 | 0.348 | 0.777 |
| 7 | 0.218 | 0.662 |
| 8 | 0.723 | 1.756 |
| 9 | 0.61 | 1.695 |
| 10 | 0.148 | 0.836 |
| 11 | 0.014 | 0.158 |
| 12 | 0.406 | 0.702 |
| 13 | 0.523 | 0.792 |
| 14 | 0.098 | 0.365 |
| 15 | 0.169 | 0.514 |
| 16 | 2.18 | 0.631 |
| 17 | 0.438 | 1.16 |
| 18 | 0.249 | 0.713 |
| 19 | 1.57 | 4.223 |
| 20 | 0.247 | 0.422 |

It is to be understood that the invention is not limited to the particular embodiments of the invention described above, as variations of the particular embodiments may be made and still fall within the scope of the appended claims.

What is claimed is:

1. A compound of Formula (I): 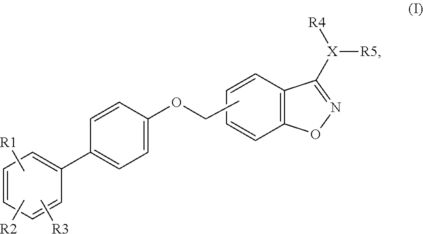
wherein:
X is O or N;
R1, R2, R3, independently of each other, is halogen, alkoxy or unsubstituted lower alkyl; and
one of R4 or R5 is hydrogen or lower alkyl, unsubstituted or mono-, bi- or tri-substituted with lower alkyl, —COOH, aryl or alkoxy, and the other is absent, when X is O, or
one of R4 or R5 is hydrogen, cycloalkyl or lower alkyl substituted with —COOH or alkoxy, and the other is hydrogen, unsubstituted lower alkyl, or lower alkyl substituted with alkoxy, cycloalkyl or heterocycloalkyl, when X is N,
or a pharmaceutically acceptable salt thereof.

2. The compound according to claim 1, wherein X is O.

3. The compound according to claim 1, wherein X is N.

4. The compound according to claim 1, wherein R1 is F or Cl.

5. The compound according to claim 1, wherein R2 is F or Cl.

6. The compound according to claim 1, wherein R3 is F, Cl, methyl or methoxy.

7. The compound according to claim 1, wherein when X is O, then one of R4 or R5 is hydrogen or lower alkyl, unsubstituted or mono-, bi- or tri-substituted with lower alkyl, —COOH, aryl or alkoxy, and the other is absent.

8. The compound according to claim 1, wherein when X is N, then one of R4 or R5 is hydrogen, cycloalkyl or lower alkyl substituted with —COOH or alkoxy, and the other is hydrogen, unsubstituted lower alkyl, or lower alkyl substituted with alkoxy, cycloalkyl or heterocycloalkyl.

9. The compound according to claim 1, wherein when X is O, then one of R4 or R5 is:
H, 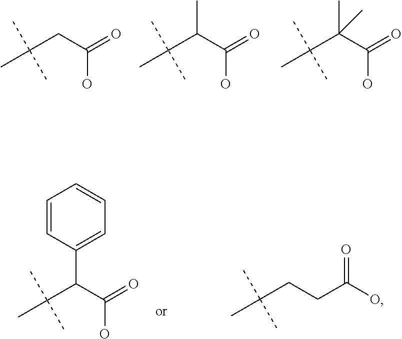
and the other is absent.

10. The compound according to claim 1, wherein when X is N, then one of R4 or R5 is H or CH₂COOH and the other is:
H, methyl, ethyl, cyclopropylmethyl,

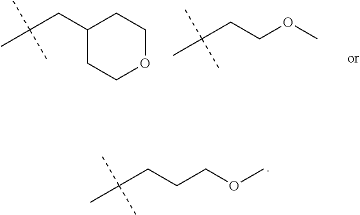

11. The compound according to claim 1, wherein said compound is:
5-(4′,5′-Difluoro-2′-methoxy-biphenyl-4-yloxymethyl)-benzo[d]isoxazol-3-ylamine;
([5-(4′,5′-Difluoro-2′-methoxy-biphenyl-4-yloxymethyl)-benzo[d]isoxazol-3-yl]-methyl-amine;
{[5-(4′,5′-Difluoro-2′-methoxy-biphenyl-4-yloxymethyl)-benzo[d]isoxazol-3-yl]-methyl-amino}-acetic acid;
5-(4′,5′-Difluoro-2′-methoxy-biphenyl-4-yloxymethyl)-benzo[d]isoxazol-3-ol;
[5-(4′,5′-Difluoro-2′-methoxy-biphenyl-4-yloxymethyl)-benzo[d]isoxazol-3-yloxy]-acetic acid;
2-[5-(4′,5′-Difluoro-2′-methoxy-biphenyl-4-yloxymethyl)-benzo[d]isoxazol-3-yloxy]-2-methyl-propionic acid;
2-[5-(4′,5′-Difluoro-2′-methoxy-biphenyl-4-yloxymethyl)-benzo[d]isoxazol-3-yloxy]-propionic acid;
[5-(4′,5′-Difluoro-2′-methoxy-biphenyl-4-yloxymethyl)-benzo[d]isoxazol-3-yloxy]-phenyl-acetic acid;
3-[5-(4′,5′-Difluoro-2′-methoxy-biphenyl-4-yloxymethyl)-benzo[d]isoxazol-3-yloxy]-propionic acid;
7-(2′,4′,5′-Trifluoro-biphenyl-4-yl-oxymethyl)-benzo[d]isoxazol-3-ol;
7-(4′,5′-Difluoro-2′-methoxy-biphenyl-4-yloxymethyl)-benzo[d]isoxazol-3-ol;
7-(2′-Chloro-4′,5′-difluoro-biphenyl-4-yloxymethyl)-benzo[d]isoxazol-3-ol;
7-(4′,5′-Difluoro-2′-methyl-biphenyl-4-yloxymethyl)-benzo[d]isoxazol-3-ol;
[5-(4′,5′-Difluoro-2′-methoxy-biphenyl-4-yloxymethyl)-benzo[d]isoxazol-3-yl]-(2-methoxy-ethyl)-amino]-acetic acid;
{[5-(4′,5′-Difluoro-2′-methoxy-biphenyl-4-yloxymethyl)-benzo[d]isoxazol-3-yl]-(3-methoxy-propyl)-amino}-acetic acid;
{[5-(4′,5′-Difluoro-2′-methoxy-biphenyl-4-yloxymethyl)-benzo[d]isoxazol-3-yl]-ethyl-amino}-acetic acid;
{Cyclopropylmethyl-[5-(4′,5′-difluoro-2′-methoxy-biphenyl-4-yloxymethyl)-benzo[d]isoxazol-3-yl]-amino}-acetic acid;
{[5-(4′,5′-Difluoro-2′-methoxy-biphenyl-4-yloxymethyl)-benzo[d]isoxazol-3-yl]-(tetrahydro-pyran-4-ylmethyl)-amino}-acetic acid;
{[7-(4′,5′-Difluoro-2′-methoxy-biphenyl-4-yloxymethyl)-benzo[d]isoxazol-3-yl]-methyl-amino}-acetic acid; or
5-(2′,4′,5′-Trifluoro-biphenyl-4-yloxymethyl)-benzo[d]isoxazol-3-ol.

12. A pharmaceutical composition, comprising a therapeutically effective amount of a compound according to claim 1 and a pharmaceutically acceptable carrier and/or adjuvant.
